(19) United States
(12) Patent Application Publication
(10) Pub. No.: US 2023/0191054 A1
(43) Pub. Date: Jun. 22, 2023

(54) DEVICE AND METHOD FOR ALTERNATELY MEASURING THORACIC PRESSURES AND FOR SEALING OESOPHAGEAL SECRETION

(71) Applicant: Creative Balloons GmbH, Waghäusel (DE)

(72) Inventor: Fred Göbel, Speyer (DE)

(21) Appl. No.: 17/924,858

(22) PCT Filed: May 17, 2021
(86) PCT No.: PCT/IB21/54222

§ 371 Date: Nov. 11, 2022

(30) Foreign Application Priority Data
May 15, 2020 (DE) 10 2020 002 932.9
Jan. 19, 2021 (DE) 10 2021 000 220.2
Jan. 19, 2021 (DE) 10 2021 000 221.0

(51) Int. Cl. A61M 16/00; A61J 15/00; A61M 16/04

(52) U.S. Cl. CPC A61M 16/024; A61J 15/0049; A61M 16/044; A61B 2090/3966

(57) ABSTRACT

The present invention relates to a device and a method for alternately measuring the thoracic and pleural pressure and for gastropharyngeal or tracheal sealing, wherein the balloon component of a tube or catheter placed in the trachea or oesophagus alternates between two filling or functional states, wherein the filling state of the balloon component in the measuring mode assumes a value of constant, defined volume during the measurement, said value corresponding to a flaccid filling state, and the filling state of the balloon in the oesophageally or tracheally sealing functional mode maintains a constant, sealing pressure specified by the user. The controller device connected to the tube unit or catheter unit ensures rapid displacement of filling medium into and out of the tube balloon or catheter balloon in the state of tracheal or oesophageal sealing, wherein the tracheally or oesophageally sealing target pressure is maintained continuously by compensating pressure fluctuations in the balloon caused by respiratory mechanics by a continuous, compensating displacement of filling volume. The user can switch between the two functional states by means of a manual switchover function or by means of a programmable, chronological cycle. In addition to the possibility of an intermittent monitoring of the respiratory mechanics and a continuous, tracheally or oesophageally sealing balloon tamponade, the balloon placed in the trachea or oesophagus allows, in both functional states, the thoracic derivation of a triggering, respiratory-mechanical signal which can trigger a ventilating stroke assisting the patient in a ventilator connected to the device. The invention also describes structural and functional options for the simultaneous derivation of a neural and/or muscular electrical signal from the diaphragm of the patient and a respiratory-mechanical signal on the basis of thoracic or pleural pressure fluctuations derived tracheally or oesophageally.

### REFERENCE TO PENDING PRIOR PATENT APPLICATIONS

This patent application is a 371 national stage entry of pending prior International (PCT) Patent Application No. PCT/IB2021/054222, filed 17 May 2021 by Creative Balloons GmbH and Fred Göbel for DEVICE AND METHOD FOR ALTERNATELY MEASURING THORACIC PRESSURES AND FOR SEALING ESOPHAGEAL SECRETION, which patent application, in turn, claims benefit of: (i) German Patent Application No. DE 10 2020 002 932.9, filed 15 May 2020, (ii) German Patent Application No. DE 10 2021 000 220.2, filed 19 Jan. 2021 and (iii) German Patent Application No. DE 10 2021 000 221.0, filed 19 Jan. 2021.

The four (4) above-identified patent applications are hereby incorporated herein by reference.

### FIELD OF THE INVENTION

The invention is directed to a device and a method for alternately, intermittently carrying out a measuring functional mode, in particular for measuring the esophageal or thoracic pressure, on the one hand, and a sealing functional mode, in particular with a dynamically adaptive, trans- or intra-esophageal seal, on the other hand, including a catheter that is provided with at least one measuring and/or sealing balloon component that alternates between two filling states, the filling state of the balloon component (i) in the measuring functional mode having a flaccid, volume-defined, static filling of the balloon, and (ii) in the sealing functional mode preferably being set in a pressure-controlled manner, in that respiratory-mechanically caused pressure fluctuations that are transferred from the thorax to the esophageally or tracheally sealing balloon are compensated for via appropriate displacements of filling medium by a controller unit connected to the catheter unit, thus continuously maintaining a sealing target pressure that is specified by the user.

### BACKGROUND OF THE INVENTION

In the machine ventilation of patients, a problem that frequently arises is the transition from a ventilation mode that is completely controlled by the therapist into an assisted ventilation mode that supports the autonomous breathing of the patient. In assisted ventilation modes, the ventilator connected to the patient senses pressure fluctuations or volume movements that arise in the tube system that is connected to the patient. If a decrease in the pressure prevailing in the inspiratory branch of the tube system or a measurable gas movement (flow) directed toward the patient occurs during the initial inspiration by the patient, the ventilator assists the breath that is initiated by the patient until a ventilation pressure, specified by the therapist, to be achieved at the end of the inspiration (end tidal) or a desired end tidal breath volume (tidal volume) is reached.

It is generally the objective of assisted ventilation to maintain to the greatest extent possible the capability of a patient for thoracic autonomous breathing in order to ensure rapid, complication-free disconnection of the patient from the ventilator or removal of the ventilation tube (extubation) if necessary. After the extubation, the patient should continuously perform sufficient respiratory work without subsequently becoming respiratory-mechanically exhausted.

To make the capability for sufficient autonomous breathing measurable and estimatable, measuring catheters are used which are positioned in the esophagus of the patient and equipped with balloon components that are generally filled in situ with air in a flaccid and tension-free manner. The so-called esophageal pressure prevailing in the esophagus corresponds approximately to the so-called intrathoracic pressure, and is used as the standard for clinically measuring same. An optimal approximation of the pressures is achieved when the balloon component of the measuring catheter is placed approximately in the region of the transition from the lower area of the esophagus to the area in the lower third of the esophagus.

The intrathoracic pressure, which is generally converted into an electrical signal outside the patient via a pressure-receiving element, may be plotted as a coordinate with respect to the respiratory gas volume (flow) that is moved by the patient and simultaneously measured by the ventilator. The respiratory work performed by the patient is then depicted as an iterative loop. The particular capability of the patient for autonomous breathing may thus be assessed over time.

### SUMMARY OF THE INVENTION

The present invention provides esophageal pressure measuring catheters with the capability of sealing the residual esophageal lumen that forms in each case around the catheter shaft, as the result of which the rising up of stomach contents into the pharynx of the patient (gastropharyngeal reflux) may be reduced or largely prevented. The so-called aspiration of gastropharyngeally rising stomach contents is one of the known causes of ventilation-associated lung inflammation. The secretion that rises up into the pharynx during aspiration passes from there into the deep airways, which facilitates the development of inflammatory pulmonary complications.

To reduce the gastropharyngeal reflux, the upper body of the patient is elevated to a certain angle, if possible, thus enabling clinically verifiable reductions in the incidence of ventilation-associated pneumonia. The present invention is intended to enable such an effect even if it is medically indicated that the patient must remain in a horizontal body position. If the thorax of the patient is already in a position with the upper body elevated, the reflux-preventive effect may be further improved by the option, made possible within the scope of the invention, of a continuous balloon tamponade of the esophageal lumen.

It is therefore desirable to be able to change back and forth between two filling states of an esophageally placed balloon element.

On the one hand there is a volume-controlled filling state in which the balloon element is filled with a predefined volume of a filling medium, and on the other hand, a pressure-controlled filling state in which the filling pressure inside the balloon element is held approximately constant.

Furthermore, the balloon on the one hand is intended to fulfill an esophageal sealing function in order to suppress or interrupt the free rising up of secretions from the stomach into the pharynx. This function may be optimally fulfilled when the balloon is controlled to a predefined filling pressure.

On the other hand, the esophageally placed balloon is intended to assume a defined filling state that allows the intrathoracic pressure to be measured, as the result of which the catheter may be used for intermittent respiratory-mechanical monitoring of the actively breathing or machine-assisted thorax. In this regard, pressure regulation would be counterproductive, since in that case only the filling pressure that is held constant would be measured, and not the intrathoracic pressure.

Therefore, the invention utilizes a switchover of the control and regulation module in such a way that the filling pressure of the balloon during a sealing state is adjusted to be as constant as possible, while in a measuring state the pressure is not continuously adjusted, but instead only a defined filling volume of a filling medium is pushed into the balloon and is then “left to itself,” in a manner of speaking, so that it is receptive to the thoracic pressure.

However, this requires a switchover between two different operating modes. It must be kept in mind that a measuring operating mode is to be repeated at certain time intervals in order to track the development of the patient's capability for autonomous breathing, and in each case to adapt the additional machine respiration so that the patient may gradually be led back to strictly autonomous breathing.

However, a manual switchover for such an adaptive modification of the machine respiration to the progressive capability of the patient for autonomous breathing requires the continuous presence of operating personnel in order to switch or program the system in each case into the correct functional mode.

This disadvantage of the prior art has resulted in the object of the invention, to find an option for allowing a ventilated patient to be gradually led to autonomous breathing over the course of his/her recovery process, without the need for constant support from operating personnel.

To achieve this object, the invention provides that a switchover between the two functional states may be triggered manually as well as via a programmable time cycle.

Such a manual switchover may be made at a controller unit that generates the pressure in the catheter balloon for the intermittent measuring function, or maintains it synchronously with the respiratory-mechanically generated pressure changes in the thorax, in the sense of continuous secretion sealing. The ventilator or the controlling unit on the one hand allows the change to be made manually, preferably with the touch of a button. It is thus possible for a medical practitioner or other operating personnel to change into the measuring functional mode, for example, at any time, and to check the present capability of the patient for autonomous breathing and manually adjust the ventilator if necessary.

On the other hand, the invention also provides an automatic switchover, the controller unit automatically switching back and forth between the two functional modes, based on a programmable time cycle. Due to such a functionality, the device according to the invention is able to check at regular time intervals, in a self-adaptive manner, the parameters for machine support for assisted respiration, and to optimize or reset them if necessary. The device according to the invention may therefore be used for prevention of pneumonia and for respiratory planning.

It has proven to be advantageous for the catheter to be a feeding catheter and/or decompression catheter that is nasogastrically or orogastrically inserted into the esophagus, or also into the duodenum or into the jejunum via the stomach.

There is an option for the sealing balloon component to tamponade or seal the entire thoracic esophagus, or to encompass only the upper half or the lower half of the thoracic esophagus.

It is recommended in the invention that the sealing and/or measuring balloon be preformed with a diameter or circumference that exceeds the diameter or circumference of the respective lumen, in particular the esophageal lumen. This results in the advantage that the lumen in question may be tamponaded tension-free, but still in a space-filling and sealing manner. Since in this regard the surface of the measuring balloon does not have to be expanded, the pressure inside the balloon element is equal to the pressure on the outside of the balloon envelope, thus in the present case the thoracic pressure in the area of the lumen, in particular esophageal lumen, in question.

Within the scope of the invention, the sealing and optionally also measuring balloon has a balloon end that is extended in the proximal direction, toward the extracorporeal catheter end, and whose diameter exceeds the outer diameter of the catheter shaft supporting the balloon, and which forms a gap via which the sealing balloon may be filled and acted on by pressure. By fastening the balloon in question only with its distal balloon end to the catheter shaft in such cases, a supply line to the balloon for filling it with a filling medium or also emptying it is obtained in a very simple manner. Moreover, a gap having a comparatively large cross section allows comparatively high flow to/from the balloon, so that dynamic, in particular respiratory-mechanically caused pressure fluctuations, may be adjusted relatively quickly, and an optimal seal is always ensured.

The segment of the balloon that forms the balloon and/or the gap may have a web-like, partially collapsing inner structure that keeps the supply line to the balloon at least partially open. A permanently open flow connection between the intracorporeal, esophageally placed balloon on the one hand and an extracorporeal pressure controller on the other hand ensures that an immediate adjustment of dynamic pressure fluctuations is possible at any time.

The measuring balloon component is to be arranged in such a way that it is situated in the lower half of the thoracic esophagus when the catheter is properly positioned, i.e., in the area of the diaphragm, where the pressure fluctuations are greatest.

In addition to an embodiment in which the same balloon is used for measuring and for sealing, it may also be provided that the sealing balloon and the measuring balloon are designed as structurally separate and separately fillable components. If these components are controllable to different pressures or filling volumes, the pressure of the sealing balloon may be continuously controlled, while the measuring balloon is continuously filled only up to a flaccid state.

There are various options for arranging a measuring balloon and a sealing balloon relative to one another. Within the scope of a first embodiment, the measuring balloon may be situated concentrically inside the sealing balloon.

On the other hand, it is also possible for the measuring balloon to be situated in series, below or distal to the sealing balloon.

Radiopaque markers on the shaft tube of the catheter, in particular in the area of the proximal and/or distal end of a balloon component, allow the length and/or position of the balloon component or balloon components in question to be made visible via X-ray. It is thus possible, if necessary, to correct or optimize the position of an esophageal catheter according to the invention inside a patient to ensure maximum sensitivity to pressure fluctuations or other signals to be recorded.

A control and/or regulation unit is connected or connectable to the measuring and/or sealing balloon components of the catheter; its task on the one hand is to coordinate the various functional modes or their sequence, and on the other hand is to be able to control the filling volume of the particular measuring balloon, in the measuring functional mode, in such a way that it assumes a flaccid, tension-free form due to incomplete, volume-defined filling, while in the sealing functional mode the filling state of the particular sealing balloon is regulated in a pressure-controlled manner.

In particular, a control and/or regulation unit according to the invention may be designed in such a way that at least three operating modes are selectable: namely, a strictly measuring functional mode, a strictly sealing functional mode, and an automatic operating mode in which the automatic controller continuously triggers a change between the measuring functional mode and the sealing functional mode, in particular based on a programmable time cycle. Thus, there are only two different functional modes, namely, either the measuring functional mode with a constant filling volume, or the sealing functional mode with a constant filling pressure. However, there is also a third operating mode in which a switch is made back and forth between these two functional modes.

For definition of the present functional state of the system according to the invention, i.e., the first or second functional mode that is selected in each case, a selection module is provided which includes at least one logical output whose output signal in one functional state is high, but in the other functional state is low. The invention profits from the fact that two possible functional states, namely, a measuring functional mode on the one hand and a sealing functional mode on the other hand, may be represented by a single digital signal, in that the logical value “high” is associated with a first functional state, and the logical value “low” is associated with the other functional state.

The selection module may be designed in the manner of a flip-flop or a bistable toggle circuit, including a setting input, which for a rising flank or for a high level of the input signal at this input sets the output signal at the logical output to “high,” and including a resetting input, which for a rising flank or for a high level of the input signal at this input sets the output signal at the logical output to “low.” Such a bistable toggle circuit thus forms a type of “memory” that remembers the most recently set functional mode in each case, and maintains this functional mode until there is a newer, different manual or machine (switchover) command.

For a manual input, it is provided that the setting input and/or the resetting input of the selection module are/is coupled to a manual input means, for example a switch or button.

On the other hand, the setting input of the selection module may be coupled to a programmable dead time or delay module that is started for a falling flank of the output signal at the logical output or for a rising flank at an inverting output, and after a programmed or programmable time interval elapses, delivers a rising flank to the setting input; and/or the resetting input is coupled to a programmable dead time or delay module that is started for a rising flank of the output signal at the logical output or for a rising flank of the output signal at the inverting output, and after a programmed or programmable time interval elapses, delivers a rising flank to the resetting input. Temporally controlled switching back and forth between two functional modes is thus possible at any time.

If multiple input signals that are associated with the same setting input or the same resetting input are linked to one another by one OR gate each, one or more input signals of at least one OR gate may be locked or unlocked by one or more logical blocking and/or enabling signals, in particular via one AND gate each. Further pursuing this concept of the invention, it may be further provided that one or more logical blocking and/or enabling signals are derived from a further input option, in particular an input button.

Moreover, the invention is preferably further characterized by dynamically adaptive, trans- or intra-esophageal secretion sealing, preferably including a [control loop], the actual value of the filling pressure in the balloon component or in a supply line thereof being detected and held as constant as possible by controlling to a predefined target value, in particular using a controller unit that is designed as an electro-pneumatic or electronic-pneumatic controller, and that in the sealing functional mode, in particular in the state of esophageal or tracheal sealing, continuously maintains a target pressure, specified by the user, inside the sealing balloon, and pressure fluctuations in the sealing balloon, in particular pressure fluctuations that are respiratory-mechanically caused, i.e., occurring in the course of the spontaneous respiration by the patient, being compensated for by appropriate displacements of filling medium into and out of the balloon in order to maintain the seal.

Further advantages may be achieved in that the controller unit, which is connected to the alternately measuring and sealing balloon component of the catheter, has at least one electronic pressure-controlling valve that sets the particular filling pressure in the balloon. This valve is used as an actuator that is acted on by the controller according to a predefined control algorithm, with the objective of holding the filling pressure inside the esophageally placeable balloon component as constant as possible.

In addition, the control and/or regulation unit according to the invention is intended to have a valve function that supplies the balloon and via which volume may be supplied to the balloon in a defined manner, as well as a valve function, parallel thereto, that discharges from the balloon and via which the volume may be withdrawn from the balloon. A constant filling volume of the balloon component may be set in this way.

According to a further design rule, one or both or all controllable valve components is/are designed as piezoelectronically operating actuators. Since only very small filling volumes are necessary for an esophageally placed balloon, solenoid valves are generally not sufficiently fine-tuned, and therefore the use of piezoelectronic actuators is preferred.

According to the invention, an arrangement is preferred in which the pressure-controlling valve has an integrated or connected sensor function that measures the filling pressure in the balloon, in particular via a sensor for the filling pressure in the balloon, the valve controlling the pressure in the balloon in such a way that a predefined filling pressure may be maintained, even continuously, when respiratory-mechanically caused pressure fluctuations occur in the balloon.

Reservoir-like components that have a positive pressure or negative pressure may optionally be provided upstream from the valves in question, or the valves are connected to one or more external pressure sources.

On the other hand, the controller may have a module that applies a defined air volume into the measuring balloon, and optionally subsequently withdraws it from the measuring balloon.

A further, preferred task of a control and regulation module according to the invention is to generate a trigger signal as early as possible for a connected ventilator. For this purpose, the stated control and regulation module is intended to have a settable function and/or module that recognize(s) the measured respiratory-mechanically caused pressure fluctuations in the thorax, in particular an initial intrathoracic pressure drop, as an indication of an incipient active respiratory excursion of the thorax. The advantage is that an esophageal pressure drop may be measured much earlier and also more reliably than a pressure drop in the ventilation tube system itself.

If the control and regulation module has recognized an initial intrathoracic pressure drop as an indication of an incipient active respiratory excursion of the thorax, based on such an intrathoracic pressure drop it may generate a trigger signal for triggering machine-assisted respiration by a ventilator.

To allow an incipient respiratory excursion of the thorax to be distinguished from an incidental pressure fluctuation, a comparator module is provided that compares the pressure signal to a magnitude of a pressure reduction that is necessary for triggering a triggering pulse for a ventilator. Such a comparator may receive at one of its inputs the pressure signal in question or a time derivative thereof, and at another of its inputs may receive a predefined or settable target value.

On the other hand, during assisted machine ventilation the effect regularly occurs that the sealing pressure is to be held as constant as possible in the esophageal balloon component, and an adjusted pressure drop is hardly discernible. Therefore, it is recommended in the invention that the control of the controller module is programmed with a latency or dead time that allows a certain pressure drop in the sealing balloon before the volume compensation that receives the target value takes place, in order to obtain the trigger option for machine-assisted respiration.

Therefore, in the event of a pressure drop in the sealing balloon, the control loop is to remain interrupted until a trigger signal for machine-assisted respiration has been generated. The adaptive sealing function may subsequently be immediately resumed.

Furthermore, the invention allows the visualized continuous thoracic pressure signal to be represented on a display device to inform a medical practitioner or some other operating personnel of the particular present state of the assisted ventilation.

In addition, one or more electrodes for receiving or deriving electrical signals of the patient may be situated at the esophageal catheter. The present invention thus describes a possible combination of an optional measuring and/or sealing esophageal balloon catheter with electrode-like components for deriving electrical signals from the diaphragm of the patient and from the structures that innervate the diaphragm. Such methods are known, for example, within the scope of so-called Edi catheter technology or neurally adjusted ventilatory assist (NAVA) ventilation methods. For appropriate placement of the deriving electrodes in the region where the esophagus passes through the diaphragm, parameters that are important for optimizing the synchronization of the ventilator and of the patient are available. Thus, for example, muscle action potentials of the diaphragm muscle may recognize the initial, early start of the patient's effort to inhale, and may already trigger machine assistance of the breathing initiated by the patient at a point in time when the patient in the connected ventilation tube system is not yet generating gas flow directed toward the patient, or the lungs of the patient have not yet expanded to an extent that triggers such a flow directed toward the patient.

The invention further provides that the electrode(s) are/is situated at the surface of the tube shaft or catheter shaft, in particular distal to the balloon element or to all balloon elements. While the sealing function of the balloon component preferably takes place in an upper area of the esophagus, the electrode(s) should be situated as close as possible to the diaphragm, i.e., distal to the balloon element(s).

Multiple electrodes, situated at the surface of the catheter shaft and distributed in the axial direction and spaced apart from one another, offer the advantage that multiple electrode signals are available which sense a larger range in the surroundings of the diaphragm and are therefore able to more reliably sense fluctuations in potential. For this purpose, it has proved advantageous to arrange multiple electrodes in an axial row one behind the other, similar to a phalanx that extends in the longitudinal direction of the esophagus, so that different phases of the potential may also be detected.

A reference electrode preferably delivers a shared reference potential that is preferably proximal or distal to all other electrode(s).

It is further recommended in the invention to arrange the electrode(s) in an area of the catheter shaft that passes through the diaphragm upon proper placement in the esophagus, since the greatest potential amplitudes naturally occur there.

The electrodes may be connectable to an extracorporeal amplifying, evaluating, and/or monitoring module via a wireless connection, such as Bluetooth, in order to transmit the optionally digitized electrode signals; however, a cable provides a less complicated option for transmitting information, each electrode preferably being individually contacted, in particular via a multicore cable having at least one core each for the individual terminal of each electrode.

Each electrode is preferably individually contacted, in particular via a multicore cable having at least one core each for the individual terminal of each electrode, so that all phases may be individually and separately evaluated.

According to one preferred refinement of the invention, the extracorporeal amplifying, evaluating, and/or monitoring module includes a module or a function for autocorrelation of the electrode signal or the electrode signals in order to recognize cyclically recurring sequences of the electrode signal or of the electrode signals, since it is possible to make repeatable statements regarding a present breathing cycle only on the basis of such cyclically recurring sequences.

Within the scope of such an implemented autocorrelation algorithm, a pattern sequence is correlated with subsequent pattern sequences, the degree of correlation or the correlation coefficient necessary for pattern recognition preferably being settable, preferably on a scale from −1 to +1, via an input element, for example via a rotary knob. Since the period between two successive breaths is not always exactly the same, for a breathing cycle it is possible to recognize typical patterns only by such an autocorrelation.

As soon as a reference pattern that is typical for a breathing cycle has been found via such an autocorrelation, a further module or a further function may determine the correlation of one or more such reference electrode signals with measured, respiratory-mechanically caused pressure fluctuations in the thorax, in particular using an initial intrathoracic pressure drop as an indicator of an incipient, active respiratory excursion of the thorax in order to identify, in the stored reference patterns, cyclically recurring sequences of one or more electrode signals as indicators of the onset of a neuromuscular breathing activity, or also relationships between two or more electrode phases that are typical for an incipient, active respiratory excursion of the thorax. The objective is to find a typical pattern course or typical relationships between multiple pattern courses, from which an incipient neuromuscular breathing activity may be deduced. This process is preferably fully automated, and thus requires no support from operating personnel.

A pattern sequence or phased pattern sequence that is identified within the scope of such an autocorrelation as typical for the onset of a neuromuscular breathing activity may be stored as a reference sequence or as a plurality of time-synchronous, phased pattern sequences, and is then available for a correlation in real time with presently measured electrode signals.

When sufficient agreement is recognized between a presently measured electrode signal and a stored reference sequence that is typical for the onset of a neuromuscular breathing activity, or between multiple presently measured electrode phases and pattern sequences that are typical for the onset of a neuromuscular breathing activity and stored in phases, an early trigger signal for triggering machine-assisted respiration is generated by a ventilator.

According to the invention, it is further provided that within the scope of the correlation algorithm, implemented in a module or as a function, between present electrode measured values on the one hand, and stored pattern sequences that are typical for the onset of a neuromuscular breathing activity on the other hand, the degree of correlation or the correlation coefficient necessary for recognizing an agreement is settable, preferably on a scale from −1 to +1, via an input element, for example via a rotary knob.

There are various options for transmitting a trigger signal, generated by the system according to the invention, for additional machine respiration on a ventilator. The least complicated approach is to output the trigger signal as a pulse signal, for example as a voltage signal having 0 V, corresponding to a low level, and 5 V as a high level, or as a current signal having 4 mA as a level and 20 mA as a high level, provided that the ventilator has a corresponding logical input.

For coupling between the control and regulation unit according to the invention on the one hand and the ventilator on the other hand via a parallel or serial interface, the trigger signal may be transferred as a short command sequence.

Such a command sequence may also be transferred wirelessly, for example via Bluetooth.

Alternatively, for this purpose the invention provides that a trigger signal generated by the system according to the invention is transferred as a pressure signal to a ventilator, in that air is discharged from a ventilation tube, leading from the ventilator to the patient, by means of a pressure relief valve that is controlled by the control and/or regulation unit according to the invention, in order to cause a pressure drop in the ventilation tube that is recognizable by the ventilator. A pressure drop, as would be caused during incipient inspiration due to the contraction of the diaphragm brought about by the patient, and which the ventilator waits for anyway, but at a much earlier point in time than would be possible if the pressure drop had to be effectuated by the patient him/herself, is thus recognizably simulated for the ventilator.

However, this pressure relief valve must be closed as soon as possible after the ventilator has initiated machine-assisted respiration, so that this breath does not escape through the pressure relief valve, but instead reaches the lungs of the patient. For this reason, a pressure sensor that is connected or connectable to the control and/or regulation unit is situated at a ventilation tube in order to signal to the control and/or regulation unit whether the ventilator has triggered machine-assisted respiration.

This sensor may also be used to sense the extent of the pressure drop that is caused by the pressure relief valve, so that it may be recognized whether the pressure drop that has occurred is sufficient for activating the ventilator. The pressure relief valve may then be briefly closed, and if an immediately subsequent pressure rise indicates that the machine-assisted respiration has in fact already been initiated, the pressure relief valve remains closed; otherwise, it may be reopened to increase the pressure drop in the ventilation tube system.

The pressure relief valve and/or the pressure sensor may be situated at a Y-shaped connecting piece where the shared ventilation tube splits off from the endotracheal tube into an inspiration tube and an expiration tube that are connected to the ventilator, or may be situated at a tubular connecting piece that is preferably directly connected to the ventilator.

The invention is further characterized by an endotracheal tube, comprising a tube body through which a lumen passes, and whose proximal end is connectable to a ventilator via one or more ventilation tubes, and comprising a cuff that encloses the tube body.

The cuff may be connected to the control and regulation unit via connecting lines, in particular via tube lines by means of which the cuff communicates with the control and regulation unit. As a result, for the control and regulation unit the option is provided to fill or (partially) empty the cuff according to a predefined, implemented algorithm.

Further pursuing this concept of the invention, a module or a function for the dynamically adaptive tracheal sealing of the cuff with respect to the trachea may be provided in the control and regulation unit, the actual value of the filling pressure in the cuff or in a supply line thereof being detected and held as constant as possible by controlling to a predefined target value. Pressure fluctuations in the cuff that are respiratory-mechanically caused, i.e., that occur during the spontaneous respiration of the patient due to appropriate displacements of filling medium into and out of the cuff, may thus be compensated for in order to also dynamically maintain the seal.

The invention may be refined by means of a signal input at the control and regulation unit for receiving data of a ventilator, in particular the volume flow moved from or to the patient and/or the pleural pressure.

This information may be combined with the information generated by the control and regulation unit itself and, for example, visually represented, preferably in the form of an iterating pie chart or as a respiratory work curve with the continuously measured thoracic or pleural pressure signal plotted with respect to the volume flow that is moved from or to the patient. A graphical display device, for example in the form of an LCD display, is suited for this purpose.

A method for switching a balloon component of a tube unit or catheter unit between two filling states, namely, (i) a first filling state of the balloon component in a measuring functional mode, the balloon component being in a flaccid state and having a filling that is statically set in a volume-defined manner, and (ii) a second filling state of the balloon component in a sealing functional mode, the filling of the balloon component being dynamically set in a pressure-controlled manner, in that pressure fluctuations that are transferred to the balloon component are compensated for by appropriate displacements of a filling medium by means of a controller unit that is connected to the catheter unit, so that a sealing target pressure that is specified by the user is continuously maintained, is characterized by a third functional mode in which an automatic controller continuously triggers a change between the measuring functional mode and the sealing functional mode, in particular based on a programmable time cycle.

On the one hand, for a selection of the measuring functional mode, after initial emptying of the balloon, an injection of a defined, specified volume of a filling medium into the balloon takes place which converts the balloon into a flaccid, unexpanded filling state of the balloon envelope.

On the other hand, for a selection of the sealing functional mode, the controlling module either supplies volume to or removes volume from the balloon in order to achieve and continuously hold a set sealing pressure target value.

The derivation of a relatively early trigger signal for triggering machine-assisted respiration, in a time-delayed manner, for example, may also be made possible by measuring or sensing a thoracic pressure fluctuation, the pressure curve being recorded by a pressure-receiving balloon or cuff placed in the esophagus or in the trachea of the patient, and converted into an electrical signal by the control and regulation unit or by the connected ventilator, visualized, and processed by its controller in a regulating manner.

The invention relates in particular to the combination of a continuous derivation of an electrical signal with the continuous or intermittent derivation of a thorax-mechanical signal. While electrical signals are not able to deliver direct information concerning the extent to which a respiratory excursion of the patient's thorax actually develops, the respiratory-mechanical success of a breathing effort may be detected via the profile of the thoracic pressure or pleural pressure, represented as a curve, analyzed for the ventilator control, and used by the user for ongoing ventilation planning. The combination of the two methods described within the scope of the invention allows in particular:
the verification of a derived electrical signal as actually being part of a mechanical diaphragm action;
the determination of the actual point in time of the transition of an electrical signal into a thoracically measurable change in the pleural pressure, and the quantitative correlation of the electrical signal strength with the intensity of the particular mechanical response to be used;
a continuous correlation of the electrical signal strength with the intensity of the respiratory-mechanical response;
an optional triggering of the connected ventilator as early as possible if no measurable mechanical diaphragm action takes place, in the sense of respiratory support of the patient as early as possible, which is then possible;
continuous respiratory-mechanical monitoring of the patient, it being possible to generate a cyclically iterating, patient-generated respiratory work curve based on the continuous measurement of the pleural pressure and of the volume flow in the ventilating tube system that is directed toward and away from the patient;
a control, it being possible for the esophageally measuring balloon to change from a measuring state into a continuously sealing filling state that suppresses or prevents the reflux of stomach contents directed toward the pharynx.

### BRIEF DESCRIPTION OF THE DRAWINGS

Further features, properties, advantages, and effects based on the invention result from the following description of preferred embodiments of the invention, with reference to the drawings. In the drawings:

FIG. 1 shows an overview of the device, comprising a catheter unit, supply lines, and connecting elements for connecting the catheter unit, as well as various functional components of a control and regulation unit;

FIG. 2a shows a schematic cross section of an esophageally positioned balloon catheter in the balloon-supporting section of the catheter shaft, the balloon assuming a flaccidly tamponading state according to the invention;

FIG. 2b shows a balloon body with a proximally (orally) directed balloon end that is formed across the shaft dimension for the coaxial filling of or pressure impingement on the balloon;

FIG. 2c shows a particular shaft profile of the catheter for ensuring an uninterrupted, continuously maintained volume flow between the esophageal balloon and an external volume reservoir or an external pressure source or volume source;

FIG. 3a shows a modified embodiment of the catheter unit with two concentrically situated esophageal balloons;

FIG. 3b shows a further embodiment of the device with two esophageal balloons arranged in series;

FIG. 4 shows a catheter unit that is supplemented by electrodes, integrated into the catheter shaft, for deriving electrical signals of the diaphragm and/or efferent nerves to the diaphragm;

FIG. 5 shows two module units, which operate in combination with the catheter unit described for FIG. 4, for visualizing and processing patient-derived electrical signals, and for synchronously monitoring the corresponding respiratory-mechanical response of the patient;

FIG. 6 shows a switchover logic system for alternately switching over into a measuring functional mode or a sealing functional mode, as the result of which the automatic unit is only interrupted, not switched off;

FIG. 7 shows another embodiment of a switchover logic system, it being possible to switch between an automatic functional mode and a manual functional mode using a selector switch, in the latter case it then being possible in turn to select a manual measuring functional mode and a manual sealing functional mode;

FIG. 8 shows a further modified embodiment of the invention, it being possible to switch directly between a strictly measuring functional mode, and a strictly sealing functional mode and an automatic functional mode, in the latter case a switch continuously being made back and forth, in a time-controlled manner, between a measuring functional mode and a sealing functional mode;

FIG. 9a shows yet a further modified embodiment of the invention, the trigger signal being transferred to the ventilation tube via a valve, and then being relayed as a pressure signal to the ventilator via this tube;

FIG. 9b shows one embodiment of the invention similar to the system from FIG. 9a, but with a different type of valve;

FIG. 10a shows the valve module from FIG. 9a in an enlarged illustration;

FIG. 10b shows the valve module from FIG. 9b in an enlarged illustration;

FIG. 11 shows a time diagram together with the pressure curve inside the ventilation tube, the filling pressure inside an esophageally placed balloon element, and the balloon pressure inside the cuff of an endotracheal tube, in each case plotted during two respiration cycles in the case of machine-assisted ventilation, the left portion of FIG. 11 illustrating the case that the machine respiration is triggered according to the pressure curve inside the ventilation tube, and the right portion of FIG. 11 illustrating that the machine respiration is triggered according to the pressure curve inside the esophageally placed balloon; and

FIG. 12 shows a time diagram corresponding to FIG. 11 together with the corresponding pressure curves, the left portion of FIG. 12 once again illustrating the case that the machine respiration is triggered according to the pressure curve inside the ventilation tube, and the right portion of FIG. 12 illustrating that the machine respiration is triggered according to the potential curves that are measured via the electrodes situated at the esophageally placed catheter.

### DETAILED DESCRIPTION OF THE PREFERRED EMBODIMENTS

The drawings illustrate the invention by way of example, based on an esophageally sealing catheter 1. However, it is emphasized that virtually all aspects of the present invention are also applicable to an endotracheal tube having a tracheally sealing balloon element in the form of a cuff.

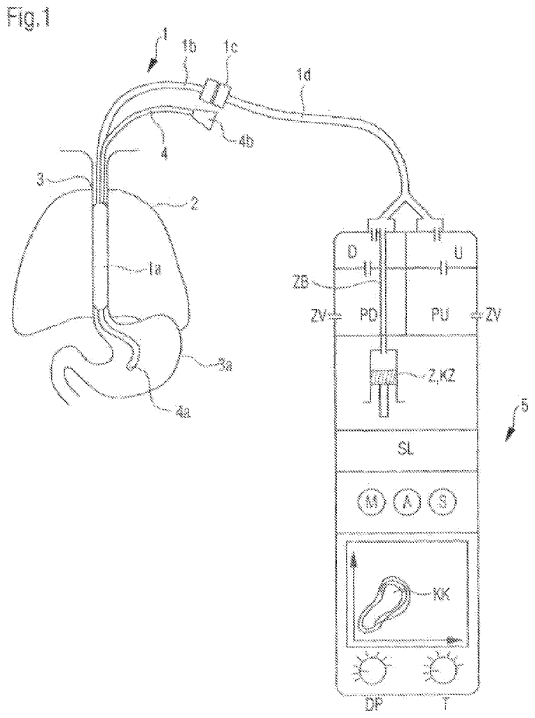

FIG. 1 shows the individual components of the device in an example of a connection that illustrates the functional principle of the invention. In the thoracic segment of the esophagus 3, the catheter unit 1 is equipped with a balloon element 1a that has already been formed to its required working dimensions during manufacture. In its preferred embodiment, the catheter itself corresponds to the typical design of a nasogastric decompression tube or feeding tube. The catheter with its distal end 4a extends into the stomach of the patient, but in alternative embodiments for so-called enteral feeding may also extend beyond the stomach into the duodenum and the jejunum. At its proximal, extracorporeal end, the supplying and discharging lumen of the catheter shaft 4 merges into a standard connector 4b for the supplying of nutritional solution and/or for the decompression or discharge of stomach contents. The catheter unit 1 at its proximal end has a tube-like connection 1b that distally merges into a feed lumen via which the balloon element 1a is filled with a preferably gaseous medium or acted on by pressure. The feed lumen may be integrated into the wall of the catheter shaft 4, for example extruded, or may also be formed as a film tube-like extension of the proximal balloon end that envelops the shaft tube. The connecting tube terminates at a connector 1c that allows, optionally via a further tube line 1d, confusion-free coupling to a controller unit 5 situated outside the body.

The tube supply line 1d from the controller 5 to the connector 1c should have a circular lumen with a diameter of at least 5 mm in order to avoid, to the greatest extent possible, flow-related pressure losses and damping effects between the balloon and the controller. Two flow- or pressure-regulating valve units D and U are connected upstream from the supply line 1d, the unit D regulating the inflow to the patient and the unit U regulating the outflow or the discharge of volume to the surroundings. The valves D and/or U are preferably based on a piezoelectronic design and mode of operation, and are therefore particularly low in noise and energy-efficient. Connected upstream from the two valves D and U are reservoir chambers PD and PU, respectively, which keep a specified positive pressure (PD) or a negative pressure (PU) as a predefined target value. The valves D and U communicate with the respective associated reservoir PD or PU. Alternatively, a positive pressure or a negative pressure may be provided via a respective connection to an external supply unit ZV.

The module 5 also includes a module Z for volume injection into the balloon element 1a of the catheter 1. A defined quantity of air may be displaced from the cylinder into the balloon element 1a or into the feed lumen 1b, 1d leading to the balloon element 1a via a piston-cylindrical arrangement KZ, for example. This is particularly important for the measuring function of the device, since the measurement per se, but in particular also the constant reproducibility of the measurement, requires flaccid filling of the balloon element 1a with a defined volume of the filling medium. The injection of the volume preferably takes place with a set specification by the controlling software of the module, but may also be variably settable by the user. Other mechanisms are possible as a nonsettable variant which ensures, for example, a spontaneously elastically straightening tube support that is installed in a rigid cylinder enclosing the tube element, the cylinder being acted on by pressure during the injection process, and thus pressing out the contents of the tube support toward the catheter balloon 1a, and automatically elastically re-straightening during the decompression of the cylinder.

At the moment of the switchover from the sealing function to the measuring function of the device, the balloon is emptied by opening the vacuum valve U. The valve U subsequently closes, and a specified quantity of a filling medium is led, via a bypass ZB, from the injecting unit Z to the input of the pressure valve D, which flows to the balloon 1a in the open state of the valve. The valve D then closes.

The valve D and/or the valve U have/has a pressure-measuring function, which in the phase of the esophageal pressure measurement continuously detects the pressure prevailing in the balloon and the supply line to the balloon, and derives it as a signal for the monitoring of the pressure curve. The measurement of the esophageal pressure preferably takes place using a gaseous medium whose volume in combination with the medium-conducting volumes of the catheter unit 1 is dimensioned such that the balloon element 1a goes into flaccid filling in order to avoid in any event an expansion of the balloon envelope that impairs the quality of the measurement. The unexpanded state of the balloon envelope ensures that any deflection of the pressure in the esophagus may be detected, or that values may be detected which, in comparison to an expanded balloon envelope, cannot be measured.

Subsequent to the measurement phase, the valve D opens and the pressure in the balloon element 1a is controlled to the sealing pressure DP that is selected by the user, and is continuously held there in the subsequent phase of the controlled sealing. The control ideally takes place as a result of the interplay between active supply and active withdrawal of filling medium to/to [sic; from] the catheter balloon 1a.

This control may take place using a programmable control, logic, and/or regulation unit, it being possible to use a higher-order control logic system SL in order to switch back and forth between a measuring functional mode FM, in which the filling state of the balloon element 1a is controlled to a constant filling volume, and a sealing functional mode FS, in which the filling state of the balloon element 1a is controlled to a constant filling pressure.

This higher-order control system SL has an input possibility with at least two options that switch the system either into the functional state FS of sealing (button S, sealing) or into the functional state FM of measuring (button M, monitoring [sic; measuring]). On the other hand, the alternation between these two functional states may also be specified automatically or by a control algorithm, for which purpose a button A (automatic unit) may be provided.

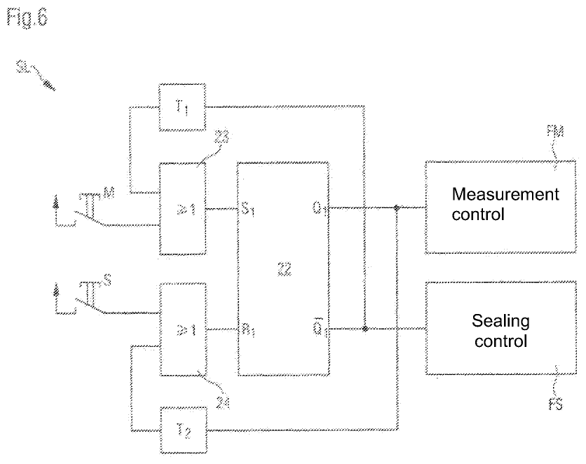

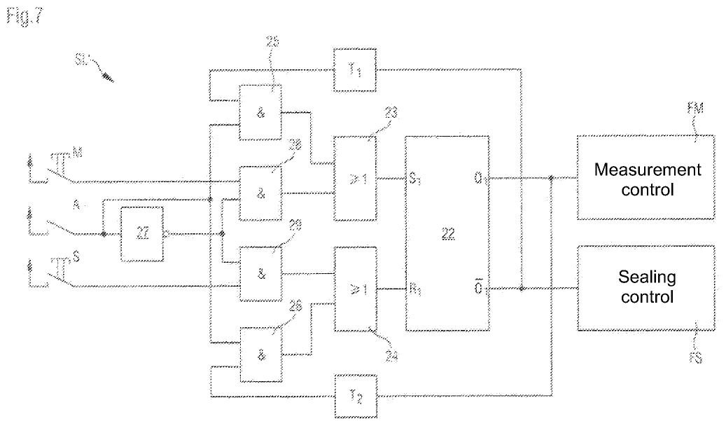

The higher-order control system SL may be designed as shown in FIG. 6, for example. For this purpose, [it] may preferably have a bistable toggle circuit 22, with a noninverting output Q1 that is set by a high level at the input S1, and that is reset [by] a high level at the input R1. The bistable toggle circuit 22 is preferably flank-triggered; i.e., the respective rising flank of the input signals at the inputs S1, R1 triggers in each case the setting or resetting operation, while the further signal pattern at the input in question has no effect until a subsequent rising flank arrives. The output Q̅1 always has the inverted signal of the output Q1.

As long as a high level is present at the output Q1, the system according to the invention operates in the measuring functional mode FM, the filling state of the balloon element 1a being controlled to a constant filling volume, while the output Q̅1 is low.

In contrast, if a high level is present at the output Q̅1, the system according to the invention operates in the sealing functional mode FS, the filling state of the balloon element 1a being controlled to a constant filling pressure, while the output Q1 is low.

The output of a first OR gate 23 is connected to the setting input S1; this first OR gate has two inputs, one of which may be connected to a high level via a button M, but otherwise has a low level. If the button M is pressed, this high level reaches the input of the OR gate 23, and from there is relayed to the setting input S1 of the bistable toggle circuit 22; the output Q1 is set to the high level, and the system immediately goes into the measuring functional mode FM.

In addition, the output of a second OR gate 24 is connected to the resetting input R1 of the bistable toggle circuit 22; this second OR gate likewise has two inputs, one of which may be connected to a high level via a button S, but otherwise has a low level. If the button S is pressed, this high level reaches the input of the OR gate 24, and from there is relayed to the resetting input R1 of the bistable toggle circuit 22; the output Q1 is set to the low level, and instead, the inverting output Q̅1 is set to the high level, and the system immediately goes into the sealing functional mode FS.

As is further apparent from FIG. 6, the inverting output Q̅1 of the bistable toggle circuit 22 is fed back to the second input of the OR gate 23 via a first timer module or delay module T1. A positive flank at the output Q̅1 of the bistable toggle circuit 22, i.e., a change from a low level to a high level, accordingly reaches the OR gate 23, delayed by a settable time T1, and from there is immediately relayed to the setting input S1 of the bistable toggle circuit 22 and then triggers an automatic change of the output signal Q1 from a low level to a high level; thus, after remaining for a time T1 in the sealing functional mode FS, the system automatically changes into the measuring functional mode FM.

In addition, there is a second feedback from the noninverting output Q1 of the bistable toggle circuit 22 to the second input of the OR gate 24 via a second timer module or delay module T2. A positive flank at the output Q1 of the bistable toggle circuit 22, i.e., a change from a low level to a high level, accordingly reaches the OR gate⁻ 24, delayed by a settable time T2, and from there is immediately relayed to the resetting input R1 of the bistable toggle circuit 22 and then triggers an automatic change of the output signal Q1 from a high level to a low level, while instead, the inverting output Q̅1 is switched over to a high level; thus, after remaining for a time T2 in the measuring functional mode FS [sic; FM], the system automatically changes into the sealing functional mode FS.

Accordingly, the switchover logic system SL from FIG. 6 shows the behavior of a permanent automatic unit that cannot be switched off; via the buttons M or S a type of temporary override function is triggered, namely, a temporally limited change into a manually selectable state which then remains active for a time interval T1 or T2; the system subsequently automatically returns to the automatic state, and switches back and forth between the two functional states FM, FS in a time-controlled manner.

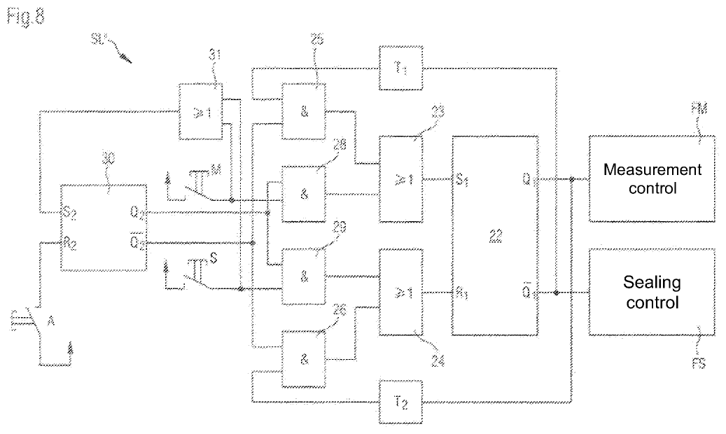

The higher-order control logic system SL′ from FIG. 7 offers the option of being able to switch the automatic unit completely off. A switch A having two stable switching states is provided for this purpose. If the switch A is closed, the system is in an automatic state; i.e., a high level at the input of the switch A reaches one input each of an AND gate 25, 26 when the switch A is closed. As a result, these two AND gates are transparent, in a manner of speaking, and respond immediately to a rising flank at their respective other input. The output signal of the time module T1 is provided at the respective other input of the AND gate 25, and, the same as for the control logic system SL, connects a rising flank through to the inverting output Q̅1, delayed by a time T1, and the output signal of the time module T2 is provided at the respective other input of the AND gate 26, and, the same as for the control logic system SL, connects a rising flank through to the noninverting output Q1, delayed by a time T2. In this automatic switching state, a time-controlled mode change, i.e., a constant, time-controlled switching back and forth between the two functional modes FM, FS, thus continuously takes place.

In contrast, if the switch A is opened, a low level is present at one input each of the two AND gates 25, 26, and the two gates 25, 26 are thus blocked; i.e., at their outputs they do not respond to the output signals of the timer modules T1, T2, and the automatic unit is switched off.

Instead, a high level now reaches one input each of two further AND gates 28, 29 via an inverting module 27, and these further AND gates now become transparent or respond sensitively to the signal at their respective other input. At this location, for the AND gate 28 a button M is connected, and for the AND gate 29 a button S is connected. Both buttons M, S have their inputs at a high level, and connect this high level through to the respective AND gate 28, 29 when the button M, S in question is manually actuated. The AND gate 28, 29 in question then likewise generates at its output a high level, which for the AND gate 28 is relayed to the OR gate 23, and for the AND gate 29 is relayed to the OR gate 24. As a result, when the button M is pressed, the output Q1 of the bistable trigger element 22 is set and the system immediately goes into the measuring functional mode FM, whereas when the button S is pressed, the inverting output Q̅1 of the bistable trigger element 22 is set to a high level and the system immediately goes into the sealing functional mode FM.

As long as the automatic unit is switched off, the system remains in the particular most recently selected functional mode FM, FS until either of the respective other functional modes FS, FM is selected, or until the automatic unit is switched on by closing the switch A.

Thus, in this control logic system SL′, each selected functional mode FM, FS, including the automatic unit, is stable until a newer input takes place. However, for manually selecting a functional mode FM, FS it is necessary to first switch off the automatic unit, and then in a second action, to select the particular functional mode FM, FS by pressing a button M, S. In contrast, directly pressing a button M, S has no effect unless the automatic unit is switched off.

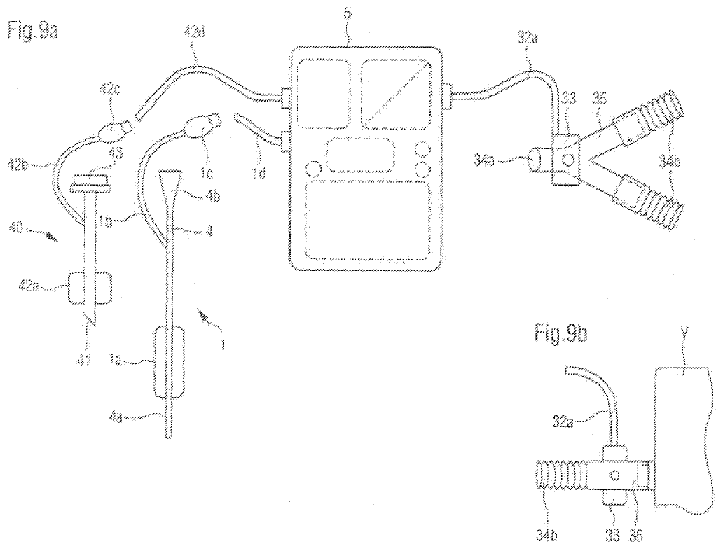

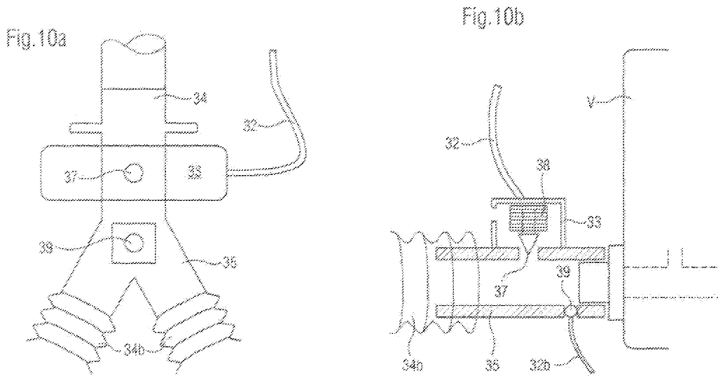

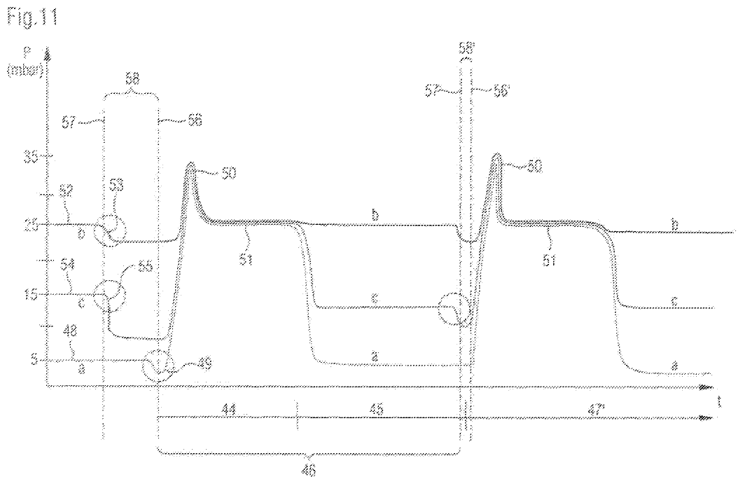

For technical laypersons, this could result in misunderstandings concerning the particular valid operating mode. To rule this out, there is a further embodiment of a higher-order control logic system SL″, illustrated in FIG. 8.

Here, the function of the switch A from FIG. 7 is transferred to a second bistable toggle circuit 30.

The noninverting output Q2 of the second bistable toggle circuit is connected to one input each of the two AND gates 28, 29, and the respective other input of the two AND gates 28, 29 is connected to the button M or to the button S. Thus, for a high level at the output Q2, the AND gates 28, 29 are transparent, and by pressing a button M or S, via the downstream OR gates 23, 24 either the setting input S1 is activated in order to select the functional mode FM, or the resetting input R1 is activated in order to select the functional mode FS.

On the other hand, the inverting output Q̅2 of the bistable trigger element 30 is simultaneously set to a low level, and the two AND gates 25, 26 connected to this inverting output Q̅2 are thus blocked, and therefore prevent automatic operation from taking place via the timer modules T1, T2.

As is further apparent in FIG. 8, the output of a further OR gate 31 is connected to the setting input S2 of the bistable trigger element 30, and the two inputs of this further OR gate are respectively connected to the output of the switch M and to the output of the switch S. When one of the buttons M or S is actuated, a rising flank thus always reaches the setting input S2 of the bistable trigger element 30, and brings it into the above-described state with high levels at the output Q2, [in] which the two AND gates 28, 29 may in turn become transparent.

As long as the button A is not pressed, the bistable trigger element 30 cannot be reset, and remains in this state, which may be referred to as manual operation and in each case one of the two manually selectable functional modes FM or FS being carried out, it being possible at any time to switch between these two functional modes FM, FS by pressing the respective other button S, M.

In contrast, if the button A is pressed, the bistable trigger element 30 is reset, and a high level is then present at the inverting output Q̅2, so that the two AND gates 25, 26 connected thereto are now transparent and respond to the flanks at the outputs Q1, Q̅1 of the bistable trigger element 22, which are delayed via the timer modules T1, T2, so that a switch is continuously made back and forth between the two functional modes FM, FS, which corresponds to the automatic unit operation.

In other words, pressing a button M, S, A immediately results in the respective operating mode FM or FS or the automatic operating mode, and the particular operating mode remains active until some other button M, S, A is pressed.

The monitoring of the measured pressure values may take place in various ways. The pressure signal is displayed as a continuous absolute value, for example. It may also be displayed in combination with the volume (flow) that is actively moved by the patient, as an iterating loop KK, to make the respiratory work of the patient representable over time. In addition, the so-called transpulmonary pressure, which results from subtracting the pleural pressure from the so-called alveolar pressure, may be determined.

As a further application option, the unit may also be utilized in both functional states for triggering machine-assisted respiration. Corresponding deflections of the intrathoracic or pleural pressure are temporally accompanied by the start of mechanical respiration of the patient's thorax, even before measurable movements of respiratory gas occur in the tube system connected to the patient. The user specifies a certain thoracic or pleural pressure drop, to be generated by the patient, as a trigger threshold, it being possible for the particular pressure difference to be set via a rotary knob or control knob T, for example in a stepless or rastered manner.

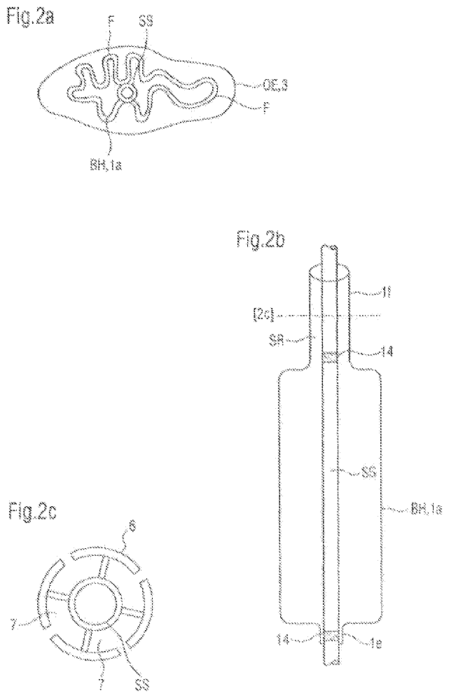

FIG. 2a schematically shows a balloon catheter 1 according to the invention in the functional state of the tamponading esophageal seal. The lumen of the esophagus OE, in the shown cross section of the organ, is illustrated as an inwardly folded, star-shaped space F. The balloon envelope BH, which is already completely formed during manufacture, lies without tension against the fold in the organ mucosa in the manner of a loosely overlying jacket. An expansion of the balloon envelope is to be avoided, in particular in the functional state of a fairly long-term esophageally sealing balloon tamponade, since the exposed mucosa on the one hand are very sensitive to pressure, and on the other hand, an irritating bolus perception by the patient should be avoided.

For the combined measuring and sealing balloon 1a described, the invention proposes an approximately cylindrically formed balloon body having a diameter of 15 to 35 mm, preferably 25 to 30 mm. The length is 6 to 12 cm, preferably 8 to 10 cm. The balloon 1a is to be made of a thin-walled material having low volume expansibility. Polyurethanes having a Shore hardness of 90 A to 95 A or 55 D are preferably used. The wall thickness of the balloon body 1a is in the range of 5 to 30 μm, preferably 10 to 15 μm. The sealing pressures, which are set to avoid gastropharyngeal reflux in the balloon 1a, are typically in a range of 5 to 30 mbar, preferably in a range of 15 to 25 mbar.

FIG. 2b shows a particular formation of the combined esophageal sealing and measuring balloon 1a, which at the distal end 1e facing the stomach is fixed to the shaft tube SS, and at the proximal end 1f is tapered in such a way that a gap SR results between the surface of the shaft tube SS and the balloon end 1f, via which the balloon may be extracorporeally filled or acted on with a filling pressure. The gap SR thus allows the balloon 1a to be filled, regardless of a filling lumen that is extruded into the wall of the shaft tube SS, which on the one hand allows particularly large cross sections of the supplying or discharging catheter lumen that opens into the digestive tract, and on the other hand allows particularly flow-efficient cross sections in the supply or discharge of the filling medium to or from the balloon 1a.

FIG. 2c shows a particular design of the catheter shaft SS in the area above the sealing balloon 1a, as a transverse section plane [2c]. The shaft tube SS is enclosed by a profile structure 6, which in the case of a force acting externally on the catheter 1, for example a peristaltically occurring contraction, leaves a residual space 7 open for the displacement of filling medium, so that an interruption in the communication of the balloon 1a with the extracorporeal controlling unit 5 may be avoided. The profile 6 is preferably made of an elastic, self-straightening material such as polyurethane. The profile extends from the proximal balloon end 1f into the area of the transition from the shaft tube to the supply line 1b. In an alternative design, the profile extends distally into the area of the sealing balloon body, or extends through to the lower fixing point of the balloon 1a on the catheter shaft SS.

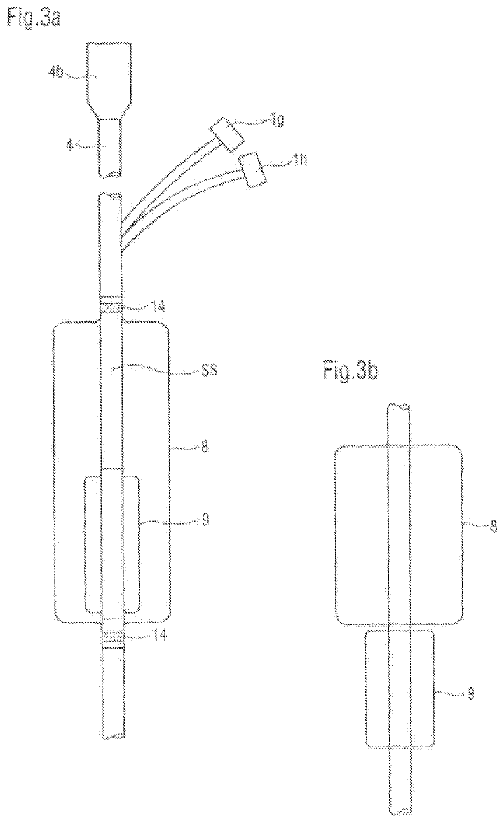

FIG. 3a shows an alternative design of the catheter unit 1 in which the catheter is equipped with two mutually concentric balloons 8, 9, and the inner balloon 9 has a measuring function and the outer balloon 8 seals the esophagus in an organ-compatible, tamponading manner. The two balloons are filled via separate supply lines 10 and 11, and are connected to a controller 5 that is modified with corresponding inlets. In this case, the connection of the supply line that fills the measuring balloon takes place directly at the volume injector Z, among other places.

The measuring balloon 9 preferably has a diameter of 8 to 12 mm, is likewise made of a soft film with a preferably low volume expansibility, and is manufactured, for example, from a PUR having a Shore hardness of 95 A. The dimensions and used materials of the sealing tamponading balloon 8 correspond to the information described above for the esophageal seal.

FIG. 3b shows an alternative, sequential arrangement of two balloon bodies, the measuring balloon 9 preferably being distally situated to allow it to be preferably placed in the transition of the lower esophagus to the middle third of the esophagus, which is preferred for detecting the thoracic pressure. The sealing balloon 8 is to be positioned in the region of the upper thoracic half of the esophagus.

The method for handling the system made up of the catheter unit 1 and the controller module 5 according to FIG. 1, optionally with characteristics of the catheter unit 1 according to one or more of FIGS. 2a through 3b, is carried out as follows:

The catheter unit 1 is typically nasogastrically positioned. The correct positioning of the tamponading sealing and measuring catheter balloon 1a between the upper and the lower sphincter muscles of the esophagus is confirmed by an X-ray of the thorax, the upper and lower ends of the balloon 1a being emphasized by appropriately contrasted markers 14 on the shaft tube SS of the catheter 1.

After the position of the balloon 1a is checked and the catheter 1 is fixed in the area of the nasal opening, the catheter is connected to the controller unit 5.

As the first function step of the controller unit 5, the valve U is opened, as the result of which the balloon body 1a is completely emptied. After the valve U is closed, a predefined volume of a filling medium is led directly to the opened valve D via a volume injection unit Z, and is displaced across the valve into the catheter balloon. The valve D closes, and via a pressure-receiving function that is preferably integrated into the valve, now measures the filling pressure prevailing in the balloon 1a, which corresponds approximately to the intrathoracic pressure, as a continuous value. A first visualization of the intrathoracic pressure then takes place, either as a continuous pressure curve or as a continuous iterating pie chart of a respiratory work diagram. The correct positioning of the balloon 1a is confirmed by a typical diagram of the esophageal pressure curve.

The user checks the continuous thoracic pressure signal for typical depressions, triggered by the resulting thoracic autonomous breathing of the patient. These depressions, when imaged sufficiently clearly, may be used for triggering machine-assisted respiration. The trigger threshold or pressure difference to be achieved may then be set by the user via a rotary controller T.

In the measuring mode, the user may observe the thoracic pressure as a continuous curve/signal, and may have iterating pressure-volume curves (respiratory work curves), or also the computed, so-called transpulmonary, pressure, represented.

The transition from the measuring mode into the sealing mode takes place via a manual switchover (button S) by the user. At this moment the pressure reservoir PD is connected to the valve P, and the negative pressure reservoir PU is connected to the valve U. Volume is now either supplied to or withdrawn from the balloon in order to reach the particular set esophageal sealing target pressure value DP or to continuously maintain it.

To obtain the trigger option for triggering machine-assisted respiration, the control by the controller may be programmed with a certain latency that allows a certain pressure drop in the balloon body before the volume displacement that is directed toward the balloon and that maintains the sealing target value occurs.

The switchover or switchback from the sealing mode to the measuring mode may be triggered by actuating the M button, or may also take place in cycles that are specified by the user.

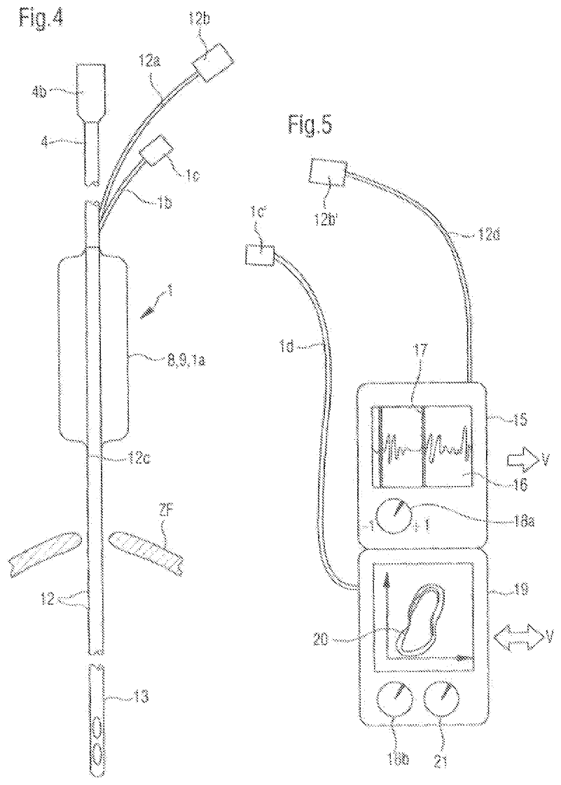

FIG. 4 shows a catheter unit 1 according to the invention which includes additional drain electrodes 12 for electrical action potentials of the diaphragm muscle ZF and/or the neural structures that innervate the diaphragm. The electrodes 12 are spaced apart from one another on the surface of the catheter shaft 4 in an axial distribution, preferably at the distal catheter end 13 distal to the balloon 1a or a balloon module 8, 9. The electrodes are situated below or distal to the esophageal balloon component 8 or 9, and in the preferred design detect the area above and also below the diaphragm. The individual electrodes 12 are led out of the catheter shaft in the area of the extracorporeal catheter end via a cable 12a that bundles all phases. The connection to the attached hardware takes place via an appropriate multipole connector 12b. For the derivation, the electrodes 12 are switched individually or as a whole to a reference electrode 12c.

The distal end 13 of the catheter is optionally designed in such a way that it opens into the stomach of the patient, or also extends through the stomach into the duodenum, or through the duodenum into the jejunum of the patient.

FIG. 5 shows an example of an arrangement of signal-recording, -processing, and -evaluating modules 15, 18 that provide the user with correspondingly modular use options of a synchronous derivation of an electrical signal and a mechanical respiration-associated signal.

An amplifying and monitoring module 15 on the one hand and a respiratory-mechanical module 19 on the other hand are illustrated. The respiratory-mechanical module 19, in addition to the functions and elements described below, may also contain the functions and elements mentioned above with regard to the controller module 5, in particular valves D and/or U, pressure reservoirs PD and/or PU, a module Z for volume injection into the balloon element 1a of the catheter 1, a control logic system SL, input elements M and S for manually selecting a measuring function on the one hand or a sealing function on the other hand, and optionally also rotary knobs DP, T for inputting an esophageally sealing target pressure value or a trigger threshold.

The amplifying and monitoring module 15 is connected to one or more electrodes 12, 12c via cables 12a, 12d and preferably a detachable plug connection 12b, 12b′, and allows the continuous visualization of the electrical diaphragm activity in the form of a continuous signal curve 16. By use of an appropriate algorithm that analyzes the signal, certain cyclically recurring segments of the signal may be recognized and identified as the effective onset of “neuromuscular” breathing activity. The point in time when the patient-generated neuromuscular activity 17 is recognized may be led to the ventilator V of the patient and may trigger assisted respiration there, which provides optimal early assistance to the spontaneous breathing effort by the patient at a point in time that precedes the effective autonomous breathing that triggers a volume flow to the patient, i.e., already in the state of “isometric” patient breathing, wherein the thoracic lumen has experienced little or no enlargement, or the elastic restoring force of the lungs is not yet overcome. This option for particularly early assistance is important for many patients. To prevent fatigue of the respiratory apparatus due to frustrating breathing efforts of the patient with no volume displacement, which generally result in resetting a patient from an assisted ventilation mode into a monitored ventilation mode, respiratory-mechanically weak patients may be weaned from the ventilator more quickly, with better efficiency and targeted ventilation planning.

The signal recognition or the computation and triggering of a trigger pulse may take place using an autocorrelation algorithm, for example, that correlates a sample action with subsequent actions. The degree of correlation or the correlation coefficient necessary for a triggering may be set, preferably on a scale from −1 to +1, by the user by manual input on an input element such as a rotary knob 18a.

Parallel to the electrical signal, a mechanical signal is derived from the thorax of the patient, the thoracic pressure prevailing at the time being recorded in each case via the esophageal balloon 8, 9, 1a, and this information being led to the respiratory-mechanical module 19 via one or more tube-like supply lines 1b, 1d and preferably via a detachable plug or screw connection 1c, 1c′. The thoracic or pleural pressure curve is represented as a continuous pressure curve, for example, in this respiratory-mechanical module 19. The curve allows the user to track the progression of the thoracic capability of the patient for spontaneous respiration.

Relative deflections of the pressure curve into the negative region may be interpreted by an identifying, correlating algorithm as the start of mechanical respiration action and transmitted to the ventilator V as a trigger pulse. The signal recognition or the computation and deflection of a trigger pulse may take place using an autocorrelation algorithm, for example, that correlates a pattern course of the pressure curve with subsequent signal patterns of the pressure curve. The degree of correlation or the correlation coefficient necessary for a triggering may be set, preferably on a scale from −1 to +1, by the user by manual input on an input element such as a rotary knob 18b.

In addition to a continuous representation of the pleural pressure, the pleural pressure may be plotted as a function of the volume flow moved by the patient, and visualized as an iterating pie chart or as a respiratory work curve 20 in the respiratory-mechanical module 19. The number of iterations of the respiratory work curve 20 to be represented on the display may be manually input by the user on an input option such as an input rotary knob 21.

The respiratory-mechanical module 19 interacts with the ventilator V in both directions; i.e., it receives present measured flow values from the ventilator V, and transmits controlling or triggering pulses to the ventilator.

The described combination of electrical and mechanical signals allows in particular the correlation of neuromuscular electrical activity with effective, mechanically performed respiratory work, and on the one hand permits the user to identify that an electrical signal is related to a mechanical response. On the other hand, the evaluating algorithm can correlate the particular signal intensities of the two signals with one another. An electrical signal may also be differentiated into a supplying, motor-efferent neural signal and the subsequent muscle action potential. The user may also verify whether a neurally efferent electrical signal is transformed into a muscle action potential, or may determine the intensity of the potential. Similarly, the user may determine whether, and with what intensity, a muscle action potential is transformed into a mechanical contraction of the diaphragm muscle.

In all preferred embodiments of the balloon catheter 1, the shaft tube SS is provided with radiopaque markers 14 that make the upper and lower ends of the esophageally positioned balloon 1a or of the balloon arrangement 1a, 8, 9 visible in the X-ray image. In principle, the sealing effect of the balloon 1a, 8 should occur in the entire area between the upper and the lower esophagus sphincter. The positioning of the preferably ring-shaped markers 14 on the shaft tube SS should then correspond approximately to the respective sphincters.

Moreover, the invention describes a method for machine ventilation of patients that minimizes reflux and prevents pneumonia, it being possible for the user to change from an esophageal dynamically sealing mode into an esophageal statically measuring mode in the course of the ventilation.

Furthermore, the invention describes a method for the alternating esophageal measuring application and esophageally sealing application to a catheter unit 1, the detection of neuromuscular electrical signals of the diaphragm of the patient being made possible via an electrode arrangement 12 situated transdiaphragmally or near the diaphragm.

Accordingly, the catheter unit 1 has a structural combination of an esophageally positioned measuring and/or sealing catheter balloon 1a and electrical drain electrodes 12.

The method for handling the system made up of the catheter unit 1 and the modules 15, 18 according to FIG. 4 is carried out as follows:

The catheter unit 1 is typically nasogastrically positioned. The correct positioning of the tamponading sealing and measuring catheter balloon 1a between the upper and the lower sphincter muscles of the esophagus is confirmed by an X-ray of the thorax, the upper and lower ends of the balloon 1a being emphasized by appropriately contrasted markers 14 on the shaft tube SS of the catheter 1. The probe-like catheter 1 has the functions of a nasogastric feeding catheter, and allows the gastric decompression as well as the gastric feeding of the patient.

The drain electrodes 12 positioned distal to the balloon component 1a are preferably positioned in such a way that they come to rest on both sides of the diaphragm, i.e., transdiaphragmally.

After the position of the balloon 1a is checked and the catheter 1 is fixed in the area of the nasal opening, the drain electrodes 12 are connected to the amplifying and monitoring module 15 via the cable supply line 12a, 12b, 12b′, and 12d, for example, and the balloon 1a, 8, 9 is connected to the respiratory-mechanical module 19 via the tube supply line 1b, 1c, 1c′, and 1d.

A summed potential of multiple individual electrodes 12 or also a signal of one or more individual electrodes 12 may then be depicted in the display of the monitoring module 15 as a continuous signal curve 16. The derivation takes place relative to the signal of a reference electrode 12c that is likewise situated on the catheter shaft SS. By comparing multiple potential cycles, a module-integrated control algorithm determines the earliest possible signal identification spike 17 within the signal 16, and the specific morphology of this identification spike is correlated with the cyclically following potentials. The precision of the correlation may be set by the user by inputting a correlation coefficient that is necessary for recognizing the signal spike. If such a sample spike is recognized in a signal, the module sends a triggering pulse to the ventilator V that is connected to the patient, as the result of which the ventilator is informed of an incipient electrical diaphragm activity. The trigger pulse may be used by the ventilator V for triggering respiration that assists the breathing effort of the patient.

The respiratory-mechanical module 19 visualizes the course of the thoracic or pleural pressure in a display, either as a continuous curve or as an iterating loop. A continuous loop is created in that the ventilator V continuously determines the flow of respiratory gas to and from the patient, and leads this information as a corresponding electronic signal, for example as a voltage curve, to the respiratory-mechanical module 19, which plots the information as a function of the continuously determined thoracic pressure.

The combination of both modules 15, 19 allows, in a manner that is optimal for ventilation planning by the user, the start of a muscular action (diaphragmal action potential) to be connected to the start of an associated respiratory-mechanically active contraction of the diaphragm, and an associated deflection or depression of the thoracic pressure, in a correlating manner. In particular, based on a triggering by a potential that is derived by the diaphragm, a volume support that assists the breathing or the inspiration effort of the patient may already be initiated, even if the patient has developed little or no mechanical breathing effort. This is crucial in particular for patients who are not able, via their autonomous breathing, to generate a sufficient depression of the thoracic pressure in order to overcome the particular elasticity of the patient's lungs, or to expand the lungs in the thorax to the extent that a volume flow directed toward the patient results inside the ventilating tube system. By use of the described method, such patients may be put into an assisted ventilation mode, and continuously held there and supportively ventilated without repeated fatigue of the respiratory muscles.

As an alternative to the “early” triggering by an electrical signal, the user may change to a triggering by a “late” thoracic-mechanical signal, the trigger signal being determined from a specified settable deflection or depression of the thoracic pressure from thoracic resting pressure. Depending on the specification of the pressure deflection that is necessary for triggering the signal, the patient may make a fairly large self-contribution to achieve a certain breathing volume. The specification thus allows optimized “training of the respiratory apparatus” without the patient experiencing respiratory fatigue and having to quit the assisted autonomous breathing.

If the respiratory-mechanical module 19 does not already integrate or have the functionalities and elements of the controller module 5, in parallel or as an alternative to a connection of the catheter balloon 1a to a respiratory-mechanical module 19, the tube-like supply line 1b to the catheter balloon 1a may also be connected to a module 5 which displays the thoracic pressure curve, and which, in addition to the option of intermittently measuring the thoracic pressure, also offers the option of a continuous pressure regulation in the catheter balloon 1a with a sealing tamponading action, wherein the sealing balloon pressure, regulating in a dynamic manner, compensates for the thoracic pressure fluctuations caused by the autonomous breathing of the patient. With such a combination of the modules, continuous triggering of a ventilator assisting the patient respiration may take place via an action potential of the diaphragm, regardless of a pressure situation that has primarily a sealing effect and that is controlled by a target value, and/or regardless of an esophageal measuring function in the esophageal balloon. In the sense of respiratory training or respiratory planning, the point in time when the ventilator is triggered may once again be predefined with a certain time offset, settable by the user, for using an electrical diaphragm signal.

FIG. 9a illustrates, based on a further example, how the trigger signal that is generated by the control and regulation unit 5 may be transferred to a ventilator V.

For this purpose, an adapter 33 is connected to the control and regulation unit 5 via a cable 32a, the adapter being connected to a ventilation tube 34a of the ventilator V, for example to a Y-shaped connecting piece 35 as illustrated in FIG. 9a, which on the one hand is connected or connectable to the proximal end of the ventilation tube 34a leading to the patient, and which on the other hand may be connected to two separate tubes 34b for inspiration and expiration.

In one embodiment according to FIG. 9b, the adapter 33 is situated directly at a tubular connecting piece 36, which is directly connectable to the ventilator V.

The main component of the adapter 33 is a pressure relief valve 37 that is opened and closed by a magnet 38 that is controlled by the control and regulation unit 5 via the cable 32a.

As soon as a trigger signal has been generated by this control and regulation unit 5, i.e., machine-assisted respiration is requested by the ventilator control and regulation unit 5, this trigger signal must be communicated to the ventilator V. For this purpose, the trigger signal, optionally in a sufficiently amplified form, is switched to the magnet 38 via the cable 32a, and causes the magnet to open the pressure relief valve 37. Air may thus escape from the mutually communicating ventilation tubes 34a, 34b, and/or from the Y-shaped distributing [sic; connecting] piece 35 or from the tubular connecting piece 36. The resulting pressure drop in the ventilation tube 34b leading to the ventilator V is sensed by the ventilator V and interpreted as an attempt by the patient to lift his/her thorax in order to draw air into the lungs by means of negative pressure, and the ventilator V then triggers the desired machine-assisted respiration.

The pressure relief valve 37 is to remain open only until the desired machine-assisted respiration has been triggered. The pressure relief valve 37 is to be subsequently closed as quickly as possible so that the positive pressure generated by the ventilator V does not escape, but instead reaches the lungs of the patient. Therefore, it is further provided according to the invention that in the area of the pressure relief valve 37 a pressure sensor 39 is situated, which is connected to the control and regulation unit 5 via a cable 32b and which allows the control and regulation unit to recognize an increasing pressure in the ventilation tube 34b due to the now active ventilator V, and to immediately close the pressure relief valve 37.

In the arrangement according to FIG. 9a, the catheter unit 1 from FIG. 1, which has no electrodes, as well as the catheter unit 1 according to FIG. 4 may optionally be used, electrodes 12, 12c being situated in the area of the distal end 4a of the catheter shaft. These electrodes 12, 12c are then connected, via a cable connection 12a, 12b, 12d, to the control and regulation unit 5, which then preferably likewise has the functionality of the monitoring module 15 and of the respiratory-mechanical module 19 or is connectable to modules having this functionality. In such cases, the trigger signal may then be derived not only from the esophageal pressure inside the balloon element 1a, but also from the signals of the electrodes 12, 12c, which tap the activity potential of the diaphragm directly from the patient.

FIG. 9a also illustrates the ventilation tube or endotracheal tube 40, which includes the actual tube 41 as well as a cuff 42a that encloses it. The ventilation tube 34a may be connected to the proximal or extracorporeal end 43 of the ventilation tube 40.

The cuff 42a of the ventilation tube 40 is also subject to a sealing problem similar to that of the balloon element 1a of the esophageal catheter 1. This sealing problem is based on the fact that during a breathing cycle of the patient, the intrathoracic pressure undergoes regular fluctuations, which in particular during a temporary pressure reduction may result in the cuff 42a as well as the balloon element 1a no longer being completely seal-tight.

To minimize this effect, the invention, the same as for the esophageally placed balloon element 1a, also provides adaptive pressure regulation for the cuff 42a of the ventilation tube 40 so that the cuff 42a is continuously seal-tight over the entire breathing cycle, without resulting in atraumatic impairment when it remains in the patient for an extended period.

In other words, the pressure inside the cuff 42a is measured, either directly in the cuff 42a itself or in a supply line 42b, 42c, 42d thereof, and this measured pressure is then adjusted as closely as possible to a predefined target value by the control and regulation unit 5. This may involve the same control algorithm as for the esophageally placed balloon element 1a, with the sole difference that no switchover to a measuring functional mode is necessary for the cuff 42a.

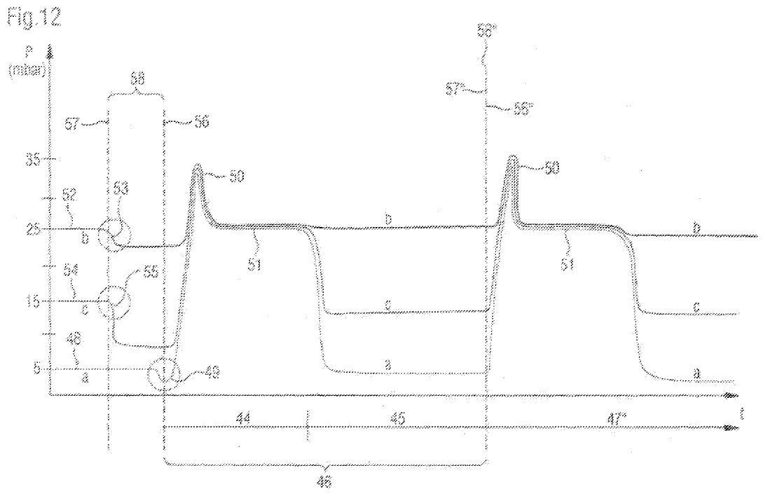

Various operating principles of the invention are depicted in FIGS. 11 and 12. In both figures, the curve a in each case represents the temporal pressure curve inside the ventilation tube 34a, 34b during machine-assisted ventilation, which is measured by the ventilator V during the inspiration phase 44 and the expiration phase 45 of a breathing cycle 46, 47′, 47″, or also influenced by same. The pressure is plotted along the ordinate in mbar as a function of the time axis t as the abscissa.

Whereas the breathing cycle 46 takes place in each case via conventional triggering by the ventilator V, for the breathing cycle 47′ a triggering takes place based on the pressure curve inside the esophageally placed balloon element 1a, and during the breathing cycle 47″ a triggering takes place based on the potential curve at the diaphragm ZF, which is measured by means of electrodes 12, 12c at the shaft 4a of the esophageally placed catheter 1.

All breathing cycles 46, 47′, 47″ share the common feature that at the end of a complete, preceding expiration phase 45, the pressure inside the ventilation tube 34a, 34b has dropped to an approximately constant value 48, which is referred to as the positive end expiratory pressure (PEEP) and is approximately +5 mbar.

For the conventional triggering method, as soon as the patient, consciously or unconsciously, has the need for a further breathing cycle 46, an appropriate stimulus reaches the diaphragm ZF via the phrenic nerve. This autonomous breathing capability, which in any case is present for the patient at least to some extent, then begins to contract. After a certain time, it deforms in an approximately conical manner, with simultaneous enlargement of the pleural cavity. As soon as the pleural cavity has noticeably enlarged, the pressure inside the ventilation tube system 34a, 34b drops slightly according to curve a. This pressure drop 49 is referred to as the initial respiratory pressure drop (IRPD). As soon as this pressure drop 49 has reached a range of approximately 2 to 3 mbar below the positive end expiratory pressure level 48, it is recognized by the ventilator V and interpreted as the desire of the patient for an inspiration phase 44, and the ventilator V now increases the pressure in the ventilation tube system 34a, 34b in order to press additional air into the lungs of the patient. In the process, the pressure in the ventilation tube system 34a, 34b increases steeply according to curve a up to a peak pressure value 50 (PEAK), which is typically approximately 35 mbar. With increasing filling of the lungs, this value drops to an elevated inspiratory pressure plateau (PLATEAU) 51, which is approximately 25 mbar. This is once again followed by an expiration phase 45, while the curve a returns once again to the original [positive] end expiratory pressure (PEEP) level 48.

Concurrently with the pressure curve a inside the ventilation tube system 34a, 34b according to curve a, the pressure curve b is measured in the cuff 42 of the endotracheal tube 40. This pressure curve has reached a constant pressure value 52 of approximately 25 mbar, for example, at the end of an expiration phase 45. As soon as the diaphragm ZF begins to contract, an onset of patient breathing (OPB) is discernible as a slight pressure drop 53 inside the cuff 42. The pressure drop 53 is only approximately 2 to 3 mbar below the initial, constant pressure value 52 of approximately 25 mbar. For the machine-assisted respiration, this pressure drop 53 remains approximately constant until the ventilator V becomes active and air is pressed into the lungs. In the process, the cuff pressure b also increases approximately to the PEAK value 50, and then follows the pressure curve a inside the ventilation tube system 34a, 34b up to the elevated inspiratory pressure plateau 51 (PLATEAU), which is already close to the initial pressure value 52 of the curve b of approximately 25 mbar, which the curve c ultimately once again seeks to attain in the expiration phase 45.

In a similar manner, the pressure curve c may be measured concurrently with the pressure curves a and b in the esophageally placed balloon element 1a of the catheter unit 1. This pressure curve has reached a constant pressure value 54 of approximately 15 mbar, for example, at the end of an expiration phase 45. As soon as the diaphragm ZF begins to contract, once again the onset of patient breathing OPB is discernible as a pressure drop 55 inside the esophageal balloon element 1a. However, the pressure drop 55 at the curve c is much more strongly pronounced than for the curve b, and is typically approximately 6 to 7 mbar below the initial constant pressure value 54 of approximately 15 mbar. During the machine-assisted respiration, this pressure drop 55 remains approximately constant or drops slightly further until the ventilator V becomes active and air is pressed into the lungs. The pressure c in the esophageally placed balloon element 1a also increases approximately to the peak value 50 of the curve a, i.e., to approximately 45 mbar, and then follows the pressure curve a inside the ventilation tube system 34a, 34b up to the elevated inspiratory pressure plateau 51, PLATEAU at approximately 25 mbar, to ultimately return to the initial pressure value 52 of approximately 15 mbar in the expiration phase 45.

Since the pressure drop 55 inside the esophageally placed balloon element 1a at the onset of patient breathing OPB is much more strongly pronounced than the approximately simultaneous pressure drop 53 inside the cuff 42a at the endotracheal tube 40, this pressure drop 55 may be more easily and quickly recognized by the control and/or regulation unit 5 according to the invention than the pressure drop 53 in the cuff 42a, and may be used to generate a trigger signal for the ventilator V.

The left portion of FIG. 11 illustrates a ventilation cycle 46 for a conventional triggering for the pressure drop 49, IRPD in the ventilation tube system 34a, 34b. This pressure drop 49, IRPD is recognized by the ventilator V at the point in time 56, whereupon the machine-assisted respiration 44 is initiated or triggered.

As is apparent in the left portion of FIG. 11, a considerable time interval 58 has elapsed since the start 57 of the contraction of the diaphragm Z (onset of breathing muscular action, BMO).

In comparison, in the method according to the right portion of FIG. 11, a pressure drop 49 of the pressure curve a in the ventilation tube system 34a, 34b is not awaited, and instead the triggering takes place based on the pressure drop 55 in the esophageally placed balloon element 1a according to the curve c. This is very pronounced, and may therefore be reliably used as the basis for generating a trigger pulse. It is apparent that this trigger point in time 56′ is much closer to the start 57 of the contraction of the diaphragm ZF than the trigger point in time 56 that is determined by the ventilator V in a conventional manner. The time interval 58′ between the start 57, BMO of the muscular activity of the diaphragm ZF and the assisting connection of the ventilator V is therefore much shorter than the corresponding time interval 58 for the conventional triggering of the ventilator.

As yet a further case, FIG. 12 illustrates the triggering based on output signals of electrodes 12, 12c at the shaft 4a of the esophageally placed catheter 1.

Since the esophagus 3, OE passes through the diaphragm ZF at the esophageal hiatus, the electrodes 12 may come into direct contact with the diaphragm ZF in order to measure its electrical muscle activity within the scope of electromyography (EMG), in particular when the electrode phalanx 12 at the catheter shaft 4a is positioned approximately one-half distally and one-half proximally with respect to the diaphragm ZF. Such positioning may be ensured with the aid of optional additional marker elements 14 at the catheter shaft 4a, for example at the proximal and distal ends of the electrode phalanx 12.

As a result, it is then no longer necessary at all for the diaphragm ZF to go into action in order to determine a trigger point in time 56″. This is important in particular since it is often difficult, specifically for elderly and/or feeble persons, to bring about any measurable pressure drop 49 at all in the ventilation tube system 34a, 34b via muscular contraction of the diaphragm ZF. Even the generation of the typically well perceivable pressure drop 55 in the esophageally placed balloon element 1a requires comparatively great physical exertion by very feeble patients, which additionally burdens and fatigues such patients.

For a triggering upon a detectable electrode signal that has been interpreted, by a preceding correlation with the esophageal pressure signal according to curve c, as an initiation 57″, BMO of the muscular activity of the diaphragm ZF, the trigger point in time 56″ may thus be determined before an esophageal pressure drop 55 has even occurred, namely, directly at the point in time 57″. This is apparent in FIG. 12 in that a pressure drop 49, 53, 55 is no longer discernible prior to the rising flank of all curves a through c at the start of the inspiration phase 44. In addition, the two points in time 56″, 57″ coincide, and the response time interval 58″ is equal to zero.

List of reference symbols

| | |
|---|---|
| 1 | catheter unit |
| 1a | balloon element |
| 1b | supply line |
| 1c | connector |
| 1c′ | connector |
| 1d | supply line |
| 1e | facing end |
| 1f | proximal end |
| 1g | connector |
| 1h | connector |
| 2 | thorax |
| 3 | esophagus |
| 3a | stomach |
| 4 | catheter shaft |
| 4a | distal end |
| 4b | connector |
| 5 | controller unit |
| 6 | profile structure |
| 7 | residual space |
| 8 | outer balloon |
| 9 | measuring balloon |
| 10 | supply line |
| 11 | supply line |
| 12 | electrodes |
| 12a | cable |
| 12b | connector |
| 12b′ | connector |
| 12c | reference electrode |
| 12d | cable |
| 13 | distal catheter end |
| 14 | marker |
| 15 | monitoring module |
| 16 | signal curve |
| 17 | identification spike |
| 18a | input rotary knob |
| 18b | input rotary knob |
| 19 | respiratory-mechanical module |
| 20 | respiratory work curve |
| 21 | input rotary knob |
| 22 | bistable toggle circuit |
| 23 | OR gate |
| 24 | OR gate |
| 25 | AND gate |
| 26 | AND gate |
| 27 | NOT gate |
| 28 | AND gate |
| 29 | AND gate |
| 30 | bistable toggle circuit |
| 31 | OR gate |
| 32a | cable |
| 32b | cable |
| 33 | adapter |
| 34a | ventilation tube |
| 34b | ventilation tube |
| 35 | Y-shaped connecting piece |
| 36 | tubular connecting piece |
| 37 | pressure relief valve |
| 38 | magnet |
| 39 | pressure sensor |
| 40 | endotracheal tube |
| 41 | tube |
| 42a | cuff |
| 42b | supply line |
| 42c | connector |
| 42d | supply line |
| 43 | proximal end |
| 44 | inspiration phase |
| 45 | expiration phase |
| 46 | breathing cycle |
| 47′ | breathing cycle |
| 47″ | breathing cycle |
| 48 | pressure level |
| 49 | pressure drop |
| 50 | peak value |
| 51 | elevated pressure plateau |
| 52 | constant pressure value |
| 53 | pressure drop |
| 54 | constant pressure value |
| 55 | pressure drop |
| 56 | trigger point in time |
| 56′ | trigger point in time |
| 56″ | trigger point in time |
| 57 | start of diaphragm activity |
| 57′ | start of diaphragm activity |
| 57″ | start of diaphragm activity |
| 58 | response time interval |
| 58′ | response time interval |
| 58″ | response time interval |
| a | curve |
| A | “automatic unit mode” button |
| b | curve |
| BH | balloon envelope |
| c | curve |
| D | valve unit |
| DP | sealing pressure |
| F | inward fold |
| FM | “monitoring” functional mode |
| FS | “sealing” functional mode |
| KK | loop |
| KZ | piston-cylindrical arrangement |
| M | “monitoring [sic; measuring] mode” button |
| OE | esophagus |
| PD | reservoir container |
| PU | reservoir container |
| Q1 | output |
| Q2 | output |
| R1 | resetting input |
| R2 | resetting input |
| S | “sealing mode” button |
| SL | programming unit |
| SR | gap |
| SS | shaft tube |
| S1 | setting input |
| S2 | setting input |
| T | rotary controller |
| U | valve unit |
| V | ventilator |
| Z | injecting unit |
| ZB | bypass |
| ZF | diaphragm |
| ZV | external supply |
| Z | volume injection module |

1. A device, comprising a catheter unit (1) with an esophageally placeable balloon component (1a) for alternating pressure measurement and secretion sealing in the esophagus (3, OE), the balloon component (1a) of the catheter unit (1) being switchable between two filling states, namely, (i) a first filling state of the balloon component (1a) in a measuring functional mode (FM), in particular for measuring the esophageal or thoracic pressure, the balloon component (1a) being in a flaccid state and having a filling that is statically set a volume-defined manner, and (ii) a second filling state of the balloon component (1a) in a sealing functional mode (FS), in particular for esophageal sealing, the filling of the balloon component (1a) being dynamically set in a pressure-controlled manner, in that respiratory-mechanically caused pressure fluctuations that are transferred from the thorax to the esophageally sealing balloon component (1a) are compensated for via appropriate displacements of a filling medium by a controller unit (5) connected to the catheter unit (1), so that a sealing target pressure that is specified by the user is continuously maintained, characterized in that a switchover between the two functional states (FM, FS) may be triggered manually as well as via a programmable time cycle.

2. The device according to claim 1, characterized in that the catheter (1) is a feeding catheter and/or decompression catheter that is nasogastrically or orogastrically insertable into the esophagus (3, OE), or also into the duodenum or into the jejunum via the stomach (3a).

3. The device according to claim 1, characterized in that the sealing balloon component (1a, 8) tamponades or seals the entire thoracic esophagus (3, OE), or encompasses only the upper half or the lower half of the thoracic esophagus (3, OE).

4. The device according to claim 1, characterized in that the sealing balloon (1a, 8) is preformed with a diameter or circumference that exceeds the diameter or circumference of the respective lumen, in particular the esophageal lumen, and thus allows a tension-free, space-filling tamponade of the lumen.

5. The device according to claim 1, characterized in that the sealing and optionally also measuring balloon (1a, 8, 9) has a balloon end that is extended in the proximal direction, toward the extracorporeal catheter end, and whose diameter exceeds the outer diameter of the catheter shaft (4, SS) supporting the balloon (1a, 8, 9), and which forms a gap (SR) via which the sealing balloon (1a, 8) may be filled and acted on by pressure.

6. The device according to claim 5, characterized in that the segment (1f) of the balloon (1a, BH) that forms the balloon (1a, BH) and/or the gap (SR) has a web-like, partially collapsing inner structure that keeps the supply line to the balloon (1a, BH) at least partially open.

7. The device according to claim 1, characterized in that the measuring balloon component (1a, 9) is positioned in the lower half of the thoracic esophagus (3, OE).

8. The device according to claim 5, characterized in that the sealing balloon (8) and the measuring balloon (9) are designed as structurally separate and separately fillable components.

9. The device according to claim 8, characterized in that the measuring balloon (9) is situated concentrically inside the sealing balloon (8).

10. The device according to claim 8, characterized in that the measuring balloon (9) is situated in series, below or distal to the sealing balloon (8).

11. The device according to claim 1, characterized by radiopaque markers (12) on the shaft tube (SS) of the catheter (1), in particular in the area of the proximal and/or distal end of a balloon component (1a, 8, 9), so that the length and/or position of the balloon component (1a) or balloon components (8, 9) in question are/is representable by an X-ray.

12. The device according to claim 1, characterized by a control and/or regulation unit (5, 15, 19, SL, SL′, SL″) for controlling and/or regulating the various functional modes, which is connected to the measuring and/or sealing balloon components (1a, 8, 9) of the catheter (1), the control and/or regulation unit (5, 15, 19, SL, SL′, SL″) being designed in such a way that in the measuring functional mode (FM), the particular measuring balloon (1a, 9) assumes a flaccid shape with incomplete, volume-defined filling, while in the sealing functional mode (FS), the filling state of the particular sealing balloon (1a, 8) is regulated in a pressure-controlled manner.

13. The device according to claim 1, characterized in that a control and/or regulation unit (5, 15, 19, SL, SL′, SL″) is designed in such a way that at least three functional modes are selectable, namely, a strictly measuring functional mode (FM), a strictly sealing functional mode (FS), and an automatic functional mode in which an automatic controller continuously triggers a change between the measuring functional mode (FM) and the sealing functional mode (FS), in particular based on a programmable time cycle.

14. The device according to claim 1, characterized by a selection module that defines the particular selected first or second functional mode (FM, FS), and that includes at least one logical output (Q1) whose output signal in one functional state is high, but in the other functional state is low.

15. The device according to claim 14, characterized in that the selection module is designed in the manner of a flip-flop or a bistable toggle circuit (22), including a setting input (S1), which for a rising flank or for a high level of the input signal at this input (S1) sets the output signal at the logical output (Q1) to “high,” and including a resetting input (R1), which for a rising flank or for a high level of the input signal at this input (R1) sets the output signal at the logical output (Q1) to “low.”

16. The device according to claim 15, characterized in that the setting input (S1) and/or the resetting input (R1) are/is coupled to a manual input means, for example a switch or button (M, S).

17. The device according to claim 15, characterized in that the setting input (S1) is coupled to a programmable dead time or delay module (T1) that is started for a falling flank of the output signal at the logical output (Q1) or for a rising flank at an inverting output (Q̅1), and after a programmed or programmable time interval (T1) elapses, delivers a rising flank to the setting input (S1).

18. The device according to claim 15, characterized in that the resetting input (R1) is coupled to a programmable dead time or delay module (T2) that is started for a rising flank of the output signal at the logical output (Q1) or for a rising flank of the output signal at the inverting output (Q̅1), and after a programmed or programmable time interval (T2) elapses, delivers a rising flank to the resetting input (R1).

19. The device according to claim 15, characterized in that multiple input signals that are associated with the same setting input (S1) or the same resetting input (R1) are linked to one another by one OR gate (23, 24) each.

20. The device according to claim 19, characterized in that one or more input signals of at least one OR gate (23, 24) are locked or unlocked by one or more logical blocking and/or enabling signals, in particular via one AND gate (25, 26, 28, 29) each.

21. The device according to claim 20, characterized in that one or more logical blocking and/or enabling signals are derived from a further input option, in particular an input button (A).

22. The device according to claim 1, characterized by dynamically adaptive, trans- or intra-esophageal secretion sealing, preferably including a control loop, the actual value of the filling pressure in the balloon component (1a) or in a supply line (1b, 1c, 1d) thereof being detected and held as constant as possible by controlling to a predefined target value, in particular using a controller unit (5) that is designed as an electro-pneumatic or electronic-pneumatic controller (5), and that in the sealing functional mode (FS), in particular in the state of esophageal sealing, continuously maintains a target pressure, specified by the user, inside the sealing balloon (1a, 8), and pressure fluctuations in the sealing balloon (1a, 8), in particular pressure fluctuations that are respiratory-mechanically caused, i.e., occurring in the course of the spontaneous respiration by the patient, being compensated for by appropriate displacements of filling medium into the balloon (1a, 8) and out of the balloon (1a, 8) in order to maintain the seal.

23. The device according to claim 1, characterized in that the controller unit (5), which is connected to the alternately measuring and sealing balloon component(s) (1a, 8, 9) of the catheter (1), has at least one electronic pressure-controlling valve (D, U) that sets the particular filling pressure in the balloon (1a, 8, 9).

24. The device according to claim 1, characterized in that the controller unit (5) has a valve function (D) that supplies the balloon (1a, 8, 9) and via which volume may be supplied to the balloon (1a, 8, 9), as well as a valve function (U), parallel thereto, that discharges from the balloon (1a, 8, 9) and via which the volume may be withdrawn from the balloon (1a, 8, 9).

25. The device according to claim 23, characterized in that one or both of the controlling valve components (D, U) are made up piezoelectronically operating control elements.

26. The device according to claim 23, characterized in that the pressure-controlling valve (D) has an integrated or connected sensor function that measures the filling pressure in the balloon (1a, 8, 9), in particular via a sensor for the filling pressure in the balloon (1a, 8, 9), the valve (D) controlling the pressure in the balloon (1a, 8, 9) in such a way that a predefined filling pressure may be maintained, even continuously, when respiratory-mechanically caused pressure fluctuations occur in the balloon.

27. The device according to claim 23, characterized in that reservoir-like components (PD, PU) that have a positive pressure or negative pressure are provided upstream from the respective valves (D, U), or the valves (D, U) are alternatively connected to one or more external pressure sources (ZV).

28. The device according to claim 23, characterized in that the controller (5) has a module (KZ) that applies a defined air volume into the measuring balloon (1a, 9), and optionally subsequently withdraws it from the measuring balloon.

29. The device according to claim 23, characterized in that the controller module (5) has a settable function (T) and/or module that recognize(s) the measured respiratory-mechanically caused pressure fluctuations in the thorax (2), in particular an initial intrathoracic pressure drop, as an indication of an incipient active respiratory excursion of the thorax (2).

30. The device according to claim 29, characterized in that the controller module (5) provides a recognized initial intrathoracic pressure drop, as an indication of an incipient active respiratory excursion of the thorax (2), as a trigger signal for triggering machine-assisted respiration by a ventilator (V).

31. The device according to claim 1, characterized by a comparator module for comparing the pressure signal to a magnitude of a pressure reduction that is necessary for triggering a triggering pulse for a ventilator (V).

32. The device according to claim 1, characterized in that the control or regulation module (5) is programmed with a latency or dead time that allows a certain pressure drop in the sealing balloon (1a) before the volume compensation that receives the target value takes place, in order to obtain the trigger option for machine-assisted respiration.

33. The device according to claim 32, characterized in that in the event of a pressure drop in the sealing balloon (1a), the control loop is interrupted until a trigger signal for machine-assisted respiration has been generated.

34. The device according to claim 1, characterized by a display device for representing the visualized, continuous thoracic pressure signal.

35. The device according to claim 1, characterized in that one or more electrodes (12, 12c) for receiving or deriving electrical signals of the patient are situated at the catheter (1).

36. The device according to claim 35, characterized in that the electrode(s) (12, 12c) are/is situated at the surface of the catheter shaft (4), in particular distal to the balloon element (1a) or to all balloon elements (8, 9).

37. The device according to claim 35, characterized in that multiple electrode(s) (12, 12c) are situated at the surface of the catheter shaft (4) and distributed in the axial direction and spaced apart from one another, preferably in an axial row one behind the other.

38. The device according to claim 35, characterized by a reference electrode (12c) that is preferably proximal or distal to all other electrode(s) (12).

39. The device according to claim 35, characterized in that the electrodes (12, 12c) are situated in an area of the catheter shaft (4) that passes through the diaphragm (ZF) upon proper placement in the esophagus (3, OE).

40. The device according to claim 35, characterized in that each electrode (12, 12c) is individually contacted, in particular via a multicore cable (12a, 12d) having at least one core each for the individual terminal of each electrode (12, 12c).

41. The device according to claim 35, characterized in that the electrodes (12, 12c) are connectable to an extracorporeal amplifying, evaluating, and/or monitoring module (15) via a cable (12a, 12d), each electrode (12, 12c) preferably being individually contacted, in particular via a multicore cable (12a, 12d) having at least one core each for the individual terminal of each electrode (12, 12c).

42. The device according to claim 41, characterized in that the extracorporeal amplifying, evaluating, and/or monitoring module (15) includes a module or a function for autocorrelation of the electrode signal or the electrode signals in order to recognize cyclically recurring sequences of the electrode signal or of the electrode signals.

43. The device according to claim 42, characterized in that within the scope of the implemented autocorrelation algorithm, a pattern sequence is correlated with subsequent pattern sequences, the degree of correlation or the correlation coefficient necessary for pattern recognition preferably being settable, preferably on a scale from −1 to +1, via an input element, for example via a rotary knob (18a).

44. The device according to claim 29, characterized by a module or a function for correlating one or more electrode signals with measured, respiratory-mechanically caused pressure fluctuations in the thorax (2), in particular using an initial intrathoracic pressure drop as an indicator of an incipient, active respiratory excursion of the thorax (2), in order to recognize cyclically recurring sequences of one or more electrode signals as indicators for the onset of a neuromuscular breathing activity.

45. The device according to claim 44, characterized in that a pattern sequence that is identified within the scope of the correlation as typical for the onset of a neuromuscular breathing activity is stored as a reference sequence and used for a correlation in real time with presently measured electrode signals, in order to generate an early trigger signal for triggering assisted respiration by a ventilator (V) when sufficient agreement is recognized between a measured electrode signal and the reference sequence.

46. The device according to claim 45, characterized in that within the scope of the implemented correlation algorithm, the degree of correlation or the correlation coefficient necessary for recognizing the onset of a neuromuscular breathing activity is settable, preferably on a scale from −1 to +1, via an input element, for example via a rotary knob (18b).

47. The device according to claim 1, characterized in that a trigger signal that is generated by the system according to the invention for additional machine respiration is transferred to a ventilator (V) as an electrical signal via one or more cables, or as a radio signal.

48. The device according to claim 1, characterized in that a trigger signal that is generated by the system according to the invention is transferred to a ventilator (V) as a pressure signal, in that air is discharged from a ventilation tube (34a, 34b), leading from the ventilator (V) to the patient, by means of a pressure relief valve (37) that is controlled by the device according to the invention, in order to cause a pressure drop in the ventilation tube (34a, 34b) that is recognizable by the ventilator.

49. The device according to claim 48, characterized in that a pressure sensor (39) that is connected or connectable to the control and/or regulation unit (5) is situated at a ventilation tube (34a, 34b) in order to signal to the control and/or regulation unit (5) whether the ventilator (V) has triggered machine-assisted respiration.

50. The device according to claim 48, characterized in that the pressure relief valve (37) and/or the pressure sensor (39) are/is situated at a Y-shaped connecting piece (35) or at a tubular connecting piece (36).

51. The device according to claim 1, characterized by an endotracheal tube (40), comprising a tube body (41) through which a lumen passes, and whose proximal end is connectable to a ventilator (V) via one or more ventilation tubes (34a, 34b), and comprising a cuff (42a) that encloses the tube body (41).

52. The device according to claim 51, characterized in that the cuff (42a) is connected to the control and regulation unit (5) via connecting lines (42b, 42c, 42d).

53. The device according to claim 52, characterized in that a module or a function for the dynamically adaptive tracheal sealing of the cuff (42a) with respect to the trachea is provided in the control and regulation unit (5), the actual value of the filling pressure in the cuff (42a) or in a supply line (42b, 42c, 42d) thereof being detected and held as constant as possible by controlling to a predefined target value, in particular pressure fluctuations in the cuff (42a), in particular pressure fluctuations that are respiratory-mechanically caused, i.e., occurring in the course of the spontaneous respiration by the patient, being compensated for by appropriate displacements of filling medium into the cuff (42a) and out of the cuff (42a) in order to maintain the seal.

54. The device according to claim 1, characterized by a signal input for receiving data of a ventilator (V), in particular the volume flow moved from or to the patient and/or the pleural pressure.

55. The device according to claim 54, characterized by a display device for representing the visualized, continuous thoracic or pleural pressure signal via the volume flow that is moved from or to the patient, in the form of an iterating pie chart or as a respiratory work curve (20).

56. A method for switching a balloon component (1a) of a tube unit or catheter unit (1) between two filling states; namely, (i) a first filling state of the balloon component (1a) in a measuring functional mode (FM), the balloon component (1a) being in a flaccid state and having a filling that is statically set in a volume-defined manner, and (ii) a second filling state of the balloon component (1a) in a sealing functional mode (FS), the filling of the balloon component (1a) being dynamically set in a pressure-controlled manner, in that pressure fluctuations that are transferred to the balloon component (1a) are compensated for by appropriate displacements of a filling medium by means of a controller unit (5) that is connected to the catheter unit (1), so that a sealing target pressure that is specified by the user is continuously maintained, characterized by a third functional mode (A) in which an automatic controller continuously triggers a change between the measuring functional mode (FM) and the sealing functional mode (FS), in particular based on a programmable time cycle.

57. The method according to claim 56, characterized in that for a selection of the measuring functional mode (FM), after initial emptying of the balloon (1a), an injection of a defined, specified volume of a filling medium into the balloon (1a) takes place which converts the balloon (1a) into a flaccid, unexpanded filling state of the balloon envelope.

58. The device according to claim 56, characterized in that for a selection of the sealing functional mode (FS), the controlling module (5) either supplies volume to or removes volume from the balloon in order to achieve and continuously hold a set sealing pressure target value (DP).
